(19) United States
(12) Patent Application Publication
(10) Pub. No.: US 2025/0002936 A1
(43) Pub. Date: Jan. 2, 2025

(54) RETGC Gene Therapy

(71) Applicant: MeiraGTx UK II Limited, London (GB)

(72) Inventor: Anastasios Georgiadis, London (GB)

(21) Appl. No.: 18/578,626

(22) PCT Filed: Jul. 13, 2022
(86) PCT No.: PCT/IB2022/056458

§ 371 Date: Jan. 11, 2024

Related U.S. Application Data
(60) Provisional application No. 63/221,883, filed on Jul. 14, 2021.

(51) Int. Cl. C12N 15/86; A61K 48/00; C12N 9/88

(52) U.S. Cl. CPC C12N 15/86; A61K 48/0058; A61K 48/0075; C12N 9/88; C12Y 406/01002; C12N 2750/14143; C12N 2830/008; C12N 2830/48; C12N 2830/50

(57) ABSTRACT

Provided herein are expression constructs, viral genomes, and vectors for the expression of retinal membrane guanylyl cyclase 1 (RetGC1), as well as pharmaceutical compositions comprising the vectors disclosed herein. Also provided are methods of using the expression constructs and vectors disclosed herein, including methods of treating a retinal disease in a subject in need thereof, wherein the retinal disease is associated with one or more mutations in the GUCY2D gene, the method comprising administering to the subject a vector disclosed herein.

### FIELD OF THE DISCLOSURE

The present disclosure relates generally to the field of molecular biology and medicine. More particularly, the disclosure provides compositions and methods for gene therapy for the treatment of retinal diseases.

### BACKGROUND

Retinal membrane guanylyl cyclase (RetGC) is located in disc membranes of photoreceptor outer segments and is one of the key enzymes in photoreceptor physiology, producing a second messenger of phototransduction, cyclic guanosine monophosphate (cGMP), in mammalian rods and cones. During photoreceptor excitation and recovery, two RetGC isozymes, RetGC1 and RetGC2 (also known as GC-E and GC-F or ROSGC1 and ROSGC2, respectively), are tightly regulated by calcium feedback mediated by guanylyl cyclase-activating proteins (GCAPs).

Over 100 mutations in GUCY2D, the gene that encodes RetGC are known to cause two major diseases: autosomal recessive Leber congenital amaurosis type 1 (arLCA or LCA1) or autosomal dominant cone-rod dystrophy (adCRD). In CRD, degeneration starts in the cones and leads to loss of the central visual field due to the high presence of cones in the macula of a non-affected retina. CRD can lead to complete blindness when degeneration of rods follows those of cones. The LCA1 phenotype appears even more severe, with photoreceptor function loss and blindness emerging very early in life.

Accordingly, novel therapies for the treatment of retinal diseases associated with GUCY2D mutations (including, but not limited to LCA1 and CRD) are urgently needed.

### SUMMARY OF THE DISCLOSURE

In one aspect, the disclosure provides an expression construct comprising: (a) a promotor sequence that confers expression in photoreceptor cells, and (b) a nucleic acid sequence encoding a retinal membrane guanylyl cyclase 1 (RetGC1), wherein the nucleic acid sequence is operably linked to the promoter.

In one embodiment, the promotor sequence is a rhodopsin kinase (RK) or a cytomegalovirus (CMV) promotor sequence.

In one embodiment, the promoter sequence comprises a sequence that is at least 90% identical to SEQ ID NO:7. In one embodiment, promoter sequence comprises SEQ ID NO:7.

In one embodiment, the promoter sequence comprises a sequence that is at least 90% identical to SEQ ID NO:8. In one embodiment, promoter sequence comprises SEQ ID NO:8.

In one embodiment, the expression construct further comprises a post transcriptional regulatory element. In one embodiment, the post transcriptional regulatory comprises a woodchuck hepatitis virus post transcriptional regulatory element (WPRE). In one embodiment, the post transcriptional regulatory element comprises a sequence that is at least 90% identical to SEQ ID NO:10. In one embodiment, the post transcriptional regulatory element comprises SEQ ID NO:10.

In one embodiment, the nucleic acid sequence encoding the RetGC1 is coding sequence (cds) from a wildtype RetGC1 (GUCY2D) gene. In one embodiment, the nucleic acid sequence encoding the RetGC1 is a codon-optimized sequence. In some embodiments, the nucleic acid sequence encoding the RetGC1 comprises a sequence that is at least 90% identical to SEQ ID NO:9. In some embodiments, the nucleic acid sequence encoding the RetGC1 comprises SEQ ID NO:9. In some embodiments, the nucleic acid sequence encoding the RetGC1 comprises a sequence that is at least 90% identical to SEQ ID NO:13. In some embodiments, the nucleic acid sequence encoding the RetGC1 comprises SEQ ID NO: 13. In some embodiments, the nucleic acid sequence encoding the RetGC1 comprises a sequence that is at least 90% identical to SEQ ID NO:14. In some embodiments, the nucleic acid sequence encoding the RetGC1 comprises SEQ ID NO:14. In some embodiments, the nucleic acid sequence encoding the RetGC1 encodes a protein comprising a sequence that is at least 90% identical to SEQ ID NO:12. In some embodiments, the nucleic acid sequence encoding the RetGC1 encodes a protein comprising SEQ ID NO:12.

In one embodiment, the expression construct further comprises a polyadenylation signal. In embodiments, the polyadenylation signal comprises a bovine growth hormone polyadenylation (BGH-polyA) signal. In one embodiment, the polyadenylation signal comprises a sequence that is at least 90% identical to SEQ ID NO:11. In one embodiment, the polyadenylation signal comprises SEQ ID NO:11.

In some embodiments, the expression construct comprises a sequence that is at least 90% identical to a sequence selected from the group consisting of SEQ ID NOS:1-4. In some embodiment, the expression construct comprises a sequence selected from the group consisting of SEQ ID NOS:1-4.

In one aspect, provided is a vector comprising an expression construct disclosed herein. In embodiments, the vector is a viral vector. In one embodiment, the vector is an adeno-associated virus (AAV) vector. In one embodiment, the vector comprises a genome derived from AAV serotype AAV2. In one embodiment, the vector comprises a capsid derived from AAV7m8.

In one aspect, provided is a pharmaceutical composition comprising a vector disclosed herein and a pharmaceutically acceptable carrier.

In one aspect, provided is a method for treating a retinal disease in a subject in need thereof, wherein the retinal disease is associated with one or more mutations in the GUCY2D gene, the method comprising administering to the subject a vector or a pharmaceutical composition disclosed herein. In some embodiments, the retinal disease is cone-rod dystrophy (CRD) or Leber congenital amaurosis type 1 (LCA1). In one embodiment, the retinal disease is LCA1.

In one aspect, provided is a method of increasing expression of rod cGMP-specific 3′,5′-cyclic phosphodiesterase subunit β (PDE6β) in a subject in need thereof, the method comprising administering to the subject a vector or a pharmaceutical composition disclosed herein.

In one aspect, provided is a method of increasing cyclic guanosine monophosphate (cGMP) levels in a photoreceptor in a subject in need thereof, the method comprising administering to the subject a vector or a pharmaceutical composition disclosed herein.

In embodiments, the vector or the pharmaceutical composition is administered by intraocular injection. In embodiments, the vector or the pharmaceutical composition is injected into the central retina of the subject.

### BRIEF DESCRIPTION OF THE DRAWINGS

FIG. 1 shows a schematic of human retina showing cell layers.

FIG. 2 shows wildtype (WT) and RetGC KO iPSC-retinal organoids at week 20. Top row: Bright field images showing whole organoids with outer segment ‘brush borders’ at the peripheral rim in both WT and RetGC KO. Middle row. Cone and Rod outer and inner segments are stained with cone opsin and Rhodopsin. Synapses in the outer (OPL) and inner plexiform layer (TPL) are stained with Ribeye and VGlut. Bipolar and amacrine/ganglion cells are stained with PKCa and calretinin. RetGC is localized to the photoreceptor outer segment in WT and absent in RetGC KO organoids.

FIG. 3 shows total protein expression (Western blot) in whole WT and RetGC KO organoids from day 40 to day 220 in control and RetGC KO organoids (normalized to β tubulin).

FIG. 4 shows the design of the four transgene cassettes that are packaged into AAV 7m8 capsids. RK and CMV promoters are incorporated with the WT GUCY2D gene with or without the WPRE element and a bovine growth hormone polyadenylation (BGH-polyA) signal.

FIG. 5 shows PDE6 staining intensity in WT and transduced RetGC KO organoids. Representative images of retinal organoid outer segments stained with Rhodopsin and PDE6R.

FIG. 6 illustrates quantitative immunofluorescence for PDE6β staining intensity within rhodopsin positive outer segments. Each point represents a tile scan of an individual organoid. Staining intensity is expressed as a percentage of a WT organoid that was processed, stained and imaged on the same block.

FIG. 7 illustrates the results of a Western blot to determine protein expression of RetGC and β tubulin (housekeeping) in retinal organoids following transduction with 7m8 vectors. Shown is the ratiometric densitometry quantification of the Western blot signal for RetGC relative to β tubulin.

FIG. 8 illustrates the quantification of cGMP concentration [nM] by FRET assay. Absorbance readings were normalized to the total protein amount [ug]. WT vs RetGC knockout (non-transduced NT) organoids were compared to organoids transduced with the four vectors (n=7 embryoid bodies (EBs) for each experimental group).

### DETAILED DESCRIPTION

Provided herein are expression constructs, viral genomes, and vectors for the expression of retinal membrane guanylyl cyclase 1 (RetGC1), as well as methods of using the expression constructs, viral genomes, and vectors for treating a retinal disease associated with one or more mutations in the GUCY2D gene.

### RetGC

RetGC catalyzes the synthesis of cGMP in rods and cones of photoreceptors. As such, RetGC plays an essential role in phototransduction by mediating cGMP replenishment during the visual cycle.

During photoreceptor excitation and recovery, two RetGC isozymes, RetGC1 and RetGC2 (also known as GC-E and GC-F or ROSGC1 and ROSGC2, respectively), are tightly regulated by calcium feedback mediated by guanylyl cyclase-activating proteins (GCAPs).

The role of RetGC1 is to replenish cGMP levels after light exposure. In the dark, cGMP levels are sustained at a steady rate, keeping the cGMP-gated channels open and maintaining partial depolarization of the cells by allowing influx of the inward current. Exposure to light leads to cGMP hydrolysis and channel closure, facilitating a sharp decline in intracellular Ca²⁺ and hyperpolarization of the cells. Under low Ca²⁺ concentrations, guanylate cyclase activating proteins (GCAPs) stimulate GC1 activity resulting in cGMP synthesis, reopening of the channels, and dark state restoration.

As a light photon passes the outer segment it is captured by the opsins embedded in the membrane of the outer segments. The second messenger cGMP is a major component in the signaling steps of the visual cycle. Balance of its synthesis and degradation in the cytoplasm of the outer segment controls the signaling steps of the visual cycle. It is generated from GTP by a reaction catalyzed by RetGC. cGMP binds to channels which allow influx of Ca²⁺ ions. On light transduction the cGMP is hydrolyzed by PDE6 to GMP causing the cGMP channels to close. This inhibits the influx of Ca²⁺, which reduces in concentration as it is being flushed out of the disc membranes.

In the phototransduction cycle, photons are absorbed by rhodopsin in rods and cone opsins in cones where 11-cis retinal is converted to all trans retinal. All trans retinal activates the alpha subunit of the G protein transducin and GDP is converted to GTP in the process. The GTP generated then activates the gamma subunit of phospodiesterase 6 (PDE6) which allows it to inhibit cGMP production. This leads to the closure of cGMP gated channels, and hence stops the influx of calcium ions. The GCAPs in the dark state are bound to calcium ions, which prevent them from associating with RetGC. Release of Ca²⁺ from GCAPs in the light state allows the GCAPs to bind RetGC and produce cGMP. Parallel to this the all trans is inactivated by phosphorylation via rhodopsin kinase and binding to arrestin. G protein transducin bound GTP is converted to GDP again. Subsequently the whole cycle repeats itself.

RetGC1 is encoded by the gene GUCY2D in humans and Gucy2e in mice. RetGC2 is encoded by the gene GUCY2F in humans.

Mutations in the GUCY2D gene coding for RetGC1 lead to severe retinal diseases in humans and mainly autosomal dominant cone-rod dystrophy (adCRD) or autosomal recessive Leber congenital amaurosis type 1 (arLCA). In CRD, degeneration starts in the cones and leads to loss of the central visual field due to the high presence of cones in the macula of a non-affected retina. CRD can lead to complete blindness when degeneration of rods follows those of cones. The LCA1 phenotype appears even more severe, with photoreceptor function loss and blindness emerging very early in life. Another gene that is involved in the pathogenesis of LCA (type 12) is rd3 coding for the retinal degeneration 3 (RD3) protein, which is an effective inhibitor of GCAP-mediated activation of RetGC1 and is involved in trafficking of RetGC1 from the inner to the outer segment in photoreceptors.

A total number of 144 different GUCY2D mutations have been described. The majority (127 mutations) result in a LCA phenotype in the affected patients. While LCA-related mutations are usually recessive and null (mainly frameshift, non-sense, and splicing mutations) and can affect all domains of the RetGC enzyme, CRD mutations are mainly dominant missense and are clustered in a “hot-spot region” which corresponds to the dimerization domain, at positions between E837 and T849.

LCA1 patients present within the first year of life and are routinely described as having reduced visual acuity, reduced or nonrecordable electroretinogram (ERG) responses, nystagmus, digito-ocular signs, and apparently normal fundus. Reports on the extent of photoreceptor degeneration associated with this disease have been conflicting. Histopathological analysis of two post-mortem retinas (a 26-wk-old preterm abortus and a 12-yr-old donor) revealed signs of photoreceptor degeneration in both rods and cones. Later studies using state of the art, in-life imaging (i.e., optical coherence tomography) revealed no obvious degeneration in patients as old as 53 years of age. More up to date studies indicate that, despite a high degree of visual disturbance, LCA1 patients retain normal photoreceptor laminar architecture, except for foveal cone outer segment abnormalities and, in some patients, foveal cone loss.

In CRD, the abnormality of rod function is less severe than that of cone function and may be detected later in the course of the disease than cone dysfunction. The diagnosis is established by electrophysiological evaluation; functional results depend on the stage of the disease and the age of the individual. The diagnosis of cone-rod dystrophy may be reinforced by the demonstration of peripheral as well as central visual field loss.

### Expression Constructs

In one aspect, provided is an expression construct comprising: (a) a promotor sequence that confers expression in photoreceptor cells, and (b) a nucleic acid sequence encoding retinal membrane guanylyl cyclase (RetGC1), wherein the nucleic acid sequence is operably linked to the promoter. As used herein, “operably linked” refer to both expression control sequences (e.g., promoters) that are contiguous with the coding sequence (cds) for RetGC1 and expression control sequences that act in trans or at a distance to control the expression of RetGC1. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein processing and/or secretion.

A great number of expression control sequences, e.g., native, constitutive, inducible and/or tissue-specific, are known in the art and may be utilized to drive expression of the RetGC1 (GUCY2D) transgene, depending upon the type of expression desired. For eukaryotic cells, expression control sequences typically include a promoter, an enhancer, and a polyadenylation sequence which may include splice donor and acceptor sites. The polyadenylation sequence generally is inserted following the sequence encoding RetGC1 and before the 3′ ITR sequence. Another regulatory component of the rAAV useful in the methods disclosed herein is an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ of the sequence encoding RetGC1 in the rAAV vector.

In one embodiment, the promotor sequence comprises a rhodopsin kinase (RK) promoter sequence. In embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7. In one embodiment, the promotor sequence comprises SEQ ID NO:7.

In one embodiment, the promotor sequence comprises a cytomegalovirus (CMV) promotor sequence. In embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8. In one embodiment, the promotor sequence comprises SEQ ID NO:8.

In some embodiments, the promoter is specific to photoreceptor cells, that is, the promoter has activity in photoreceptor cells, but has reduced or no activity in other cell types.

In one embodiment, the nucleic acid sequence encoding the RetGC1 is coding sequence from a wildtype RetGC1 (GUCY2D) gene. In one embodiment, the nucleic acid sequence encoding the RetGC1 is a codon-optimized sequence. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:9. In one embodiment, the nucleic acid sequence encoding the RetGC1 comprises SEQ ID NO:9. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:13. In one embodiment, the nucleic acid sequence encoding the RetGC1 comprises SEQ ID NO:13. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:14. In one embodiment, the nucleic acid sequence encoding the RetGC1 comprises SEQ ID NO:14. In some embodiments, the nucleic acid sequence encoding the RetGC1 encodes a protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12. In some embodiments, the nucleic acid sequence encoding the RetGC1 encodes a protein comprising SEQ ID NO:12.

In one embodiment, the expression construct comprises a post transcriptional regulatory element. In one embodiment, the expression construct comprises a woodchuck hepatitis virus post transcriptional regulatory element (WPRE). In some embodiments, the post transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In one embodiment, the post transcriptional regulatory element comprises SEQ ID NO:10.

In one embodiment, the expression construct comprises a polyadenylation signal. In one embodiment, the expression construct comprises a bovine growth hormone polyadenylation (BGH-polyA) signal. In some embodiments, the polyadenylation signal comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11. In one embodiment, the polyadenylation signal comprises SEQ ID NO:11.

In one embodiment, the expression construct comprises a nucleic acid comprising one or more inverted terminal repeats (ITR). In one embodiment, the ITR sequence is derived from AAV serotype 2. In one embodiment, the 5′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:5. In one embodiment, the 5′ ITR sequence comprises SEQ ID NO:5. In one embodiment, the 3′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:6. In one embodiment, the 3′ ITR sequence comprises SEQ ID NO:6.

### Vectors

In one aspect, provided are recombinant vectors and their use for the introduction of a transgene or an expression construct into a cell. In some embodiments, the recombinant vectors comprise recombinant DNA constructs that include additional DNA elements, including DNA segments that provide for the replication of the DNA in a host cell and expression of the target gene in target cells at appropriate levels. The ordinarily skilled artisan appreciates that expression control sequences (promoters, enhancers, and the like) are selected based on their ability to promote expression of the target gene in the target cell. “Vector,” as used herein, means a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus derived sequences). A vector may also refer to a virion comprising a nucleic acid to be delivered into a host cell, either in vitro or in vivo. In some embodiments, a vector refers to a virion comprising a recombinant viral genome, wherein the viral genome comprises one or more ITRs and a transgene.

In one embodiment, the recombinant vector is a viral vector or a combination of multiple viral vectors.

In one aspect, provided is a vector comprising any of the expression constructs disclosed herein.

In one aspect, provided is a vector comprising a nucleic acid comprising (a) a promotor sequence that confers expression in photoreceptor cells, and (b) a nucleic acid sequence encoding RetGC1, wherein the nucleic acid sequence encoding RetGC1 is operably linked to the promoter.

In one embodiment, the promotor sequence comprises an RK promoter sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7. In one embodiment, the promotor sequence comprises SEQ ID NO:7.

In one embodiment, the promotor sequence comprises a CMV promotor sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8. In one embodiment, the promotor sequence comprises SEQ ID NO:8.

In some embodiments, the promoter is specific to photoreceptor cells.

In one embodiment, the nucleic acid sequence encoding the RetGC1 is coding sequence from a wildtype RetGC1 (GUCY2D) gene. In one embodiment, the nucleic acid sequence encoding the RetGC1 is a codon-optimized sequence. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:9. In one embodiment, the nucleic acid sequence encoding the RetGC1 comprises SEQ ID NO:9. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:13. In one embodiment, the nucleic acid sequence encoding the RetGC1 comprises SEQ ID NO:13. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:14. In one embodiment, the nucleic acid sequence encoding the RetGC1 comprises SEQ ID NO:14. In some embodiments, the nucleic acid sequence encoding the RetGC1 encodes a protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12. In some embodiments, the nucleic acid sequence encoding the RetGC1 encodes a protein comprising SEQ ID NO:12.

In one embodiment, the vector comprises a nucleic acid comprising a post transcriptional regulatory element. In one embodiment, the vector comprises a nucleic acid comprising a WPRE. In some embodiments, the post transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In one embodiment, the post transcriptional regulatory element comprises SEQ ID NO:10.

In one embodiment, the vector comprises a nucleic acid comprising a polyadenylation signal. In one embodiment, the vector comprises a nucleic acid comprising a BGH-polyA signal. In some embodiments, the polyadenylation signal comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11. In one embodiment, the polyadenylation signal comprises SEQ ID NO:11.

In one embodiment, the vector comprises a nucleic acid comprising one or more inverted terminal repeats (ITR). In one embodiment, the ITR sequence is derived from AAV serotype 2. In one embodiment, the 5′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:5. In one embodiment, the 5′ ITR sequence comprises SEQ ID NO:5. In one embodiment, the 3′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:6. In one embodiment, the 3′ ITR sequence comprises SEQ ID NO:6.

In some embodiments, the vector comprises a nucleic acid comprising a sequence that is at least 80%, at least 85%, at least 90%, at least 91%, at least 92%, at least 93%, at least 94%, at least 95%, at least 96%, at least 97%, at least 98%, or at least 99% identical to any of the sequences of SEQ ID NOS:1-4. In some embodiments, the vector comprises a nucleic acid comprising a sequence selected from the group consisting of SEQ ID NOS:1-4.

In one embodiment, provided is a vector comprising a nucleic acid comprising one or more of:
(a) promotor sequence comprising an RK promoter sequence;
(b) a nucleic acid sequence encoding RetGC1, wherein the nucleic acid sequence encoding RetGC1 is operably linked to the promoter;
(c) a WPRE;
(d) a BGH-polyA signal; and
(e) one or more ITRs. In some embodiments, the vector comprises two ITR sequences.

In one embodiment, provided is a vector comprising a nucleic acid comprising one or more of:
(a) promotor sequence comprising a CMV promoter sequence;
(b) a nucleic acid sequence encoding RetGC1, wherein the nucleic acid sequence encoding RetGC1 is operably linked to the promoter;
(c) a WPRE;
(d) a BGH-polyA signal; and
(e) one or more ITRs. In some embodiments, the nucleic acid comprises two ITR sequences.

In one embodiment, provided is a vector comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding RetGC1, wherein the nucleic acid sequence encoding RetGC1 is operably linked to the promoter and wherein the nucleic acid sequence encoding RetGC1 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9, SEQ ID NO:13, or SEQ ID NO:14;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding RetGC1, wherein the nucleic acid sequence encoding RetGC1 is operably linked to the promoter and wherein the nucleic acid sequence encoding RetGC1 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9, SEQ ID NO:13, or SEQ ID NO:14;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding a RetGC1 protein, wherein the RetGC1 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:12, and wherein the nucleic acid sequence encoding the RetGC1 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding a RetGC1 protein, wherein the RetGC1 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:12 and wherein the nucleic acid sequence encoding the RetGC1 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:7;
(b) a nucleic acid sequence encoding RetGC1, wherein the nucleic acid sequence encoding RetGC1 is operably linked to the promoter and wherein nucleic acid sequence encoding RetGC1 comprises SEQ ID NO:9, SEQ ID NO:13, or SEQ ID NO:14;
(c) a post transcriptional regulatory element comprising SEQ ID NO:10;
(d) a polyadenylation signal comprising a sequence SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:8;
(b) a nucleic acid sequence encoding RetGC1, wherein the nucleic acid sequence encoding RetGC1 is operably linked to the promoter and wherein nucleic acid sequence encoding RetGC1 comprises SEQ ID NO:9, SEQ ID NO:13, or SEQ ID NO:14;
(c) a post transcriptional regulatory element comprising SEQ ID NO:10;
(d) a polyadenylation signal comprising a sequence SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:7;
(b) a nucleic acid sequence encoding a RetGC1 protein, wherein the RetGC1 protein comprises SEQ ID NO:12, and wherein the nucleic acid sequence encoding the RetGC1 protein is operably linked to the promoter;
(c) a post transcriptional regulatory element comprising SEQ ID NO:10;
(d) a polyadenylation signal comprising a sequence SEQ ID NO:11; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:8;
(b) a nucleic acid sequence encoding a RetGC1 protein, wherein the RetGC1 protein comprises SEQ ID NO:12, and wherein the nucleic acid sequence encoding the RetGC1 protein is operably linked to the promoter;
(c) a post transcriptional regulatory element comprising SEQ ID NO:10;
(d) a polyadenylation signal comprising a sequence SEQ ID NO:11; and
(e) one or more ITRs. In some embodiments, the nucleic acid comprises two ITR sequences.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The Rep reading frame encodes four proteins of molecular weight 78 kD, 68 kD, 52 kD, and 40 kD. These proteins function mainly in regulating AAV replication and rescue and integration of the AAV into a host cell's chromosomes. The Cap reading frame encodes three structural proteins of molecular weight 85 kD (VP 1), 72 kD (VP2), and 61 kD (VP3), which form the virion capsid. More than 80% of total proteins in AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, preferably the Rep and/or Cap genes, but retain functional flanking ITR sequences.

In some embodiments, the viral vector is an rAAV virion, which comprises an rAAV genome and one or more capsid proteins. In some embodiments, the rAAV genome comprises an expression cassette disclosed herein.

In some embodiments, the viral vector disclosed herein comprises a nucleic acid comprising an AAV 5′ ITR and 3′ ITR located 5′ and 3′ to sequence encoding RetGC1, respectively. However, in certain embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to the sequence encoding RetGC1. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments, the pseudotyped AAV is AAV2/7m8 (i.e., an AAV containing AAV2 ITRs and AAV7m8 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In one aspect, provided is a viral genome comprising a nucleic acid comprising (a) a promotor sequence that confers expression in photoreceptor cells, and (b) a nucleic acid sequence encoding RetGC1, wherein the nucleic acid sequence encoding RetGC1 is operably linked to the promoter.

In one embodiment, the promotor sequence comprises an RK promoter sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7. In one embodiment, the promotor sequence comprises SEQ ID NO:7.

In one embodiment, the promotor sequence comprises a CMV promotor sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8. In one embodiment, the promotor sequence comprises SEQ ID NO:8.

In some embodiments, the promoter is specific to photoreceptor cells.

In one embodiment, the nucleic acid sequence encoding the RetGC1 is coding sequence from a wildtype RetGC1 (GUCY2D) gene. In one embodiment, the nucleic acid sequence encoding the RetGC1 is a codon-optimized sequence. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:9. In one embodiment, the nucleic acid sequence encoding the RetGC1 comprises SEQ ID NO:9. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:13. In one embodiment, the nucleic acid sequence encoding the RetGC1 comprises SEQ ID NO:13. In some embodiments, the nucleic acid sequence encoding the RetGC1 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:14. In one embodiment, the nucleic acid sequence encoding the RetGC1 comprises SEQ ID NO:14. In some embodiments, the nucleic acid sequence encoding the RetGC1 encodes a protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12. In some embodiments, the nucleic acid sequence encoding the RetGC1 encodes a protein comprising SEQ ID NO:12.

In one embodiment, the viral genome comprises a nucleic acid comprising a post transcriptional regulatory element. In one embodiment, the viral genome comprises a nucleic acid comprising a WPRE. In some embodiments, the post transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In one embodiment, the post transcriptional regulatory element comprises SEQ ID NO:10.

In one embodiment, the viral genome comprises a nucleic acid comprising a polyadenylation signal. In one embodiment, the viral genome comprises a nucleic acid comprising a BGH-polyA signal. In some embodiments, the polyadenylation signal comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11. In one embodiment, the polyadenylation signal comprises SEQ ID NO:11.

In one aspect, the viral genome comprises a nucleic acid comprising one or more inverted terminal repeats (ITR). In one embodiment, the ITR sequence is derived from AAV serotype 2. In one embodiment, the 5′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:5. In one embodiment, the 5′ ITR sequence comprises SEQ ID NO:5. In one embodiment, the 3′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:6. In one embodiment, the 3′ ITR sequence comprises SEQ ID NO:6.

In some embodiments, the viral genome comprises a nucleic acid comprising a sequence that is at least 80%, at least 85%, at least 90%, at least 91%, at least 92%, at least 93%, at least 94%, at least 95%, at least 96%, at least 97%, at least 98%, or at least 99% identical to any of the sequences of SEQ ID NOS:1-4. In some embodiments, the viral genome comprises a nucleic acid comprising a sequence selected from the group consisting of SEQ ID NOS:1-4.

In one embodiment, provided is a viral genome comprising a nucleic acid comprising one or more of:
(a) promotor sequence comprising an RK promoter sequence;
(b) a nucleic acid sequence encoding RetGC1, wherein the nucleic acid sequence encoding RetGC1 is operably linked to the promoter;
(c) a WPRE;
(d) a BGH-polyA signal; and
(e) one or more ITRs. In some embodiments, the viral genome comprises two ITR sequences.

In one embodiment, provided is a viral genome comprising a nucleic acid comprising one or more of:
(a) promotor sequence comprising a CMV promoter sequence;
(b) a nucleic acid sequence encoding RetGC1, wherein the nucleic acid sequence encoding RetGC1 is operably linked to the promoter;
(c) a WPRE;
(d) a BGH-polyA signal; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding RetGC1, wherein the nucleic acid sequence encoding RetGC1 is operably linked to the promoter and wherein the nucleic acid sequence encoding RetGC1 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9, SEQ ID NO:13, or SEQ ID NO:14;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding RetGC1, wherein the nucleic acid sequence encoding RetGC1 is operably linked to the promoter and wherein the nucleic acid sequence encoding RetGC1 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9, SEQ ID NO:13, or SEQ ID NO:14;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the viral genome comprises two ITR sequences.

In one embodiment, provided is viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding a RetGC1 protein, wherein the RetGC1 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:12, and wherein the nucleic acid sequence encoding the RetGC1 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding a RetGC1 protein, wherein the RetGC1 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:12 and wherein the nucleic acid sequence encoding the RetGC1 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:7;
(b) a nucleic acid sequence encoding RetGC1, wherein the nucleic acid sequence encoding RetGC1 is operably linked to the promoter and wherein nucleic acid sequence encoding RetGC1 comprises SEQ ID NO:9, SEQ ID NO:13, or SEQ ID NO:14;
(c) a post transcriptional regulatory element comprising SEQ ID NO:10;
(d) a polyadenylation signal comprising a sequence SEQ ID NO:11; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:8;
(b) a nucleic acid sequence encoding RetGC1, wherein the nucleic acid sequence encoding RetGC1 is operably linked to the promoter and wherein nucleic acid sequence encoding RetGC1 comprises SEQ ID NO:9, SEQ ID NO:13, or SEQ ID NO:14;
(c) a post transcriptional regulatory element comprising SEQ ID NO:10;
(d) a polyadenylation signal comprising a sequence SEQ ID NO:11; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:7;
(b) a nucleic acid sequence encoding a RetGC1 protein, wherein the RetGC1 protein comprises SEQ ID NO:12, and wherein the nucleic acid sequence encoding the RetGC1 protein is operably linked to the promoter;
(c) a post transcriptional regulatory element comprising SEQ ID NO:10;
(d) a polyadenylation signal comprising a sequence SEQ ID NO:11; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:8;
(b) a nucleic acid sequence encoding a RetGC1 protein, wherein the RetGC1 protein comprises SEQ ID NO:12, and wherein the nucleic acid sequence encoding the RetGC1 protein is operably linked to the promoter;
(c) a post transcriptional regulatory element comprising SEQ ID NO:10;
(d) a polyadenylation signal comprising a sequence SEQ ID NO:11; and
(e) one or more ITRs. In some embodiments, the viral genome comprises two ITR sequences.

Other viral vectors include adenoviral (AV) vectors, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Alternatively, other systems such as lentiviral vectors can be used. Lentiviral-based systems can transduce nondividing as well as dividing cells making them useful for applications targeting, for examples, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the expression cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells includes hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this disclosure are known to those with skill in nucleic acid manipulation and include genetic engineering, recombinant engineering, and synthetic techniques. See, e.g., Sambrook et al, “Molecular Cloning. A Laboratory Manual”, 2d ed., Cold Spring Harbor Laboratory, New York (1989), and Ausubel et al., Current Protocols in Molecular Biology, John Wiley & Sons, New York, 1989); and International Patent Publication No. WO 95/13598. Further, methods suitable for producing a rAAV cassette in an adenoviral capsid have been described in U.S. Pat. Nos. 5,856,152 and 5,871,982.

Briefly, in order to package the rAAV genome into a rAAV virion, a host cell is used that contains sequences necessary to express AAV rep and AAV cap or functional fragments thereof as well as helper genes essential for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection, and protoplast fusion. In one embodiment, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

The rep and cap sequences, along with their expression control sequences, may be supplied on a single vector, or each sequence may be supplied on its own vector. Preferably, the rep and cap sequences are supplied on the same vector. Alternatively, the rep and cap sequences may be supplied on a vector that contains other DNA sequences that are to be introduced into the host cells. Preferably, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this disclosure are conventional genetic engineering or recombinant engineering techniques such as those described in the references above. For example, the rAAV may be produced utilizing a triple transfection method using either the calcium phosphate method (Clontech) or Effectene reagent (Qiagen, Valencia, Calif.), according to manufacturer's instructions. See, also, Herzog et al, 1999, Nature Medic., 5(1):56-63, for the method used in the following examples, employing the plasmid with the transgene, a helper plasmid containing AAV rep and cap, and a plasmid supplying adenovirus helper functions of E2A, E4Orf6 and VA. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions are then produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art, preferably on a separate plasmid. Thereafter, the recombinant AAV virion which directs expression of the RetGC1 transgene is isolated from the cell or cell culture in the absence of contaminating helper virus or wildtype AAV.

Expression of the RetGC1 transgene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR; and the level of protein expression may be monitored by Western blotting, immunohistochemistry, enzyme-linked immunosorbent assay (ELISA), radioimmunoassay (RIA) or by the specific methods detailed below in the Examples.

Pharmaceutical composition

Provided herein are pharmaceutical compositions comprising any of the vectors disclosed herein and a pharmaceutically acceptable excipient.

The rAAV comprising the gene encoding RetGC1 is preferably assessed for contamination by conventional methods and then formulated into a pharmaceutical composition suitable for storage and/or administration to a patient.

Formulations of the vectors disclosed herein involve the use of a pharmaceutically and/or physiologically acceptable vehicle or carrier, particularly one suitable for subretinal injection, such as buffered saline or other buffers, e.g., HEPES, to maintain pH at appropriate physiological levels

The vector of the disclosure can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

If the vector is to be stored long-term, it may be frozen in the presence of glycerol.

### Methods of Treatment

Provided herein is a method of treating a retinal disease in a subject in need thereof, wherein the retinal disease is associated with one or more mutations in the GUCY2D gene, the method comprising administering to the subject a vector disclosed herein. Also provided herein is a method of treating a retinal disease in a subject in need thereof, wherein the retinal disease is associated with one or more mutations in the GUCY2D gene, the method comprising administering to the subject a pharmaceutical composition comprising a vector disclosed herein. Provided herein is a vector for use in a method of treating a retinal disease in a subject in need thereof, wherein the retinal disease is associated with one or more mutations in the GUCY2D gene. In some embodiments, the subject carries a mutation in the GUCY2D gene.

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In some embodiments, treatment success is measured by one or more of the following: visual acuity, electroretinogram (ERG) responses, reduced nystagmus, changes in digito-ocular signs, and histopathological analysis, or optical coherence tomography.

In some embodiments, the retinal disease is cone-rod dystrophy (CRD) or Leber congenital amaurosis type 1 (LCA1). In one embodiment, the retinal disease is LCA1. In one embodiment, the retinal disease is CRD.

In one aspect, provided is a method comprising:
(a) determining whether a subject carries a mutation in the GUCY2D gene; and
(b) administering a pharmaceutical composition comprising a vector disclosed herein to the subject if the subject carries a mutation in the GUCY2D gene.

### Route and Methods of Administration

In some embodiments, the vectors or the pharmaceutical compositions disclosed herein are administered by intraocular injection. In some embodiments, the vectors or the pharmaceutical compositions disclosed herein are administered by direct retinal, subretinal, or intravitreal injection. In some embodiments, the vectors or the pharmaceutical compositions disclosed herein are administered to the central retina of a subject.

The dose of a vector of the disclosure may be determined according to various parameters, especially according to the age, weight and condition of the patient to be treated, the particular ocular disorder and the degree to which the disorder, if progressive, has developed, the route of administration; and the required regimen. Again, a physician will be able to determine the required route of administration and dosage for any particular patient. An effective amount of an rAAV carrying a nucleic acid sequence encoding RetGC1 under the control of the promoter sequence desirably ranges between about 1×10⁹ to 2×10¹² rAAV genome particles or between 1×10¹⁰ to 2×10¹¹ genome particles. A “genome particle” is defined herein as an AAV capsid that contains a single stranded DNA molecule that can be quantified with a sequence specific method (such as real-time PCR). In some embodiments, the about 1×10⁹ to 2×10¹² rAAV genome particles are provided in a volume of between about 150 to about 800 μl. In some embodiments, the about 1×10¹⁰ to 2×10¹¹ rAAV genome particles are provided in a volume of between about 250 to about 500 μl. Still other dosages in these ranges may be selected by the attending physician.

The dose may be provided as a single dose, but may be repeated for the fellow eye or in cases where vector may not have targeted the correct region of the retina for whatever reason (such as surgical complication). The treatment is preferably a single permanent treatment for each eye, but repeat injections, for example in future years and/or with different AAV serotypes may be considered. As such, it may be desirable to administer multiple “booster” dosages of a pharmaceutical compositions disclosed herein. For example, depending upon the duration of the transgene within the ocular target cell, one may deliver booster dosages at 6 month intervals, or yearly following the first administration. Such booster dosages and the need therefor can be monitored by the attending physicians, using, for example, the retinal and visual function tests and the visual behavior tests known in the art. Other similar tests may be used to determine the status of the treated subject over time. Selection of the appropriate tests may be made by the attending physician. Still alternatively, the methods disclosed herein may also involve injection of a larger volume of a vector-containing solution in a single or multiple infection to allow levels of visual function close to those found in wildtype retinas.

### Additional Methods

In one aspect, provided is a method of increasing expression of rod cGMP-specific 3′,5′-cyclic phosphodiesterase subunit β (PDE6β) in a subject in need thereof, the method comprising administering to the subject a vector disclosed herein. In one aspect, provided is a method of increasing expression of rod cGMP-specific 3′,5′-cyclic phosphodiesterase subunit β (PDE6β) in a cell, the method comprising contacting the cell with a vector disclosed herein.

In one aspect, provided is a method of increasing cGMP levels in a photoreceptor in a subject in need thereof, the method comprising administering to the subject a vector disclosed herein. In one aspect, provided is a method of increasing cGMP levels in a photoreceptor in a cell, the method comprising contacting the cell with a vector disclosed herein.

### Articles of Manufacture and Kits

Also provided are kits or articles of manufacture for use in the methods described herein. In aspects, the kits comprise the compositions described herein (e.g., compositions for delivery of a RetGC1 encoding transgene) in suitable packaging. Suitable packaging for compositions (such as ocular compositions for injection) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Also provided are kits comprising the compositions described herein. These kits may further comprise instruction(s) on methods of using the composition, such as uses described herein. The kits described herein may further include other materials desirable from a commercial and user standpoint, including buffers, diluents, filters, needles, syringes, and package inserts with instructions for performing the administration of the composition or performing any methods described herein. For example, in some embodiments, the kit comprises an rAAV for the expression of a RetGC1 encoding transgene in target cells, a pharmaceutically acceptable carrier suitable for injection, and one or more of: a buffer, a diluent, a filter, a needle, a syringe, and a package insert with instructions for performing the injections.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: Generation of RetGC Knockout (KO) Organoids as an In Vitro Disease Model for Retinal Diseases Associated with Mutations in GUCY2D

To generate RetGC KO organoids, wildtype (WT) retinal organoids were harvested at several time points during development. GUCY2D mRNA and RetGC protein levels were measured by qPCR and Western blot/immunofluorescence, respectively at various time points during retinal organoid development alongside with retina specific markers. WT human fibroblast were reprogrammed, and gene edited to delete GUCY2D-RetGC using episomal reprogramming factors and CRISPR/CAS9. The KO induced pluripotent stem cell (iPSC) clones were differentiated into retinal organoids alongside their unedited (WT) isogenic control line. The presence of photoreceptor markers and the absence of RetGC protein at the expected time points in development were verified.

RetGC protein was translocated to the photoreceptor outer segment in the mammalian retina. By immunofluorescence, RetGC protein could be detected in the outer segment structures of WT organoids, where it was co-localized with Rhodopsin. Loss of RetGC protein in the mature RetGC KO organoids was confirmed by immunofluorescence and Western blot. There was also a significant reduction in GUCY2D (RetGC) mRNA.

In addition to loss of RetGC in the outer segment, phototransduction protein phosphodiesterase-6-beta (PDE6β) was found to be reduced in the outer segments of RetGC KO organoids. PDE6β has a central role in the phototransduction cycle. Upon light stimulation, cGMP is hydrolysed by PDE6β to GMP causing the cGMP channels in the outer segment disc to close, leading to hyperpolarisation of the photoreceptor cell.

The above cited properties of RetGC KO organoids showed that these organoids could be utilized as an in vitro disease model to test the efficacy of RetGC viral vectors to restore protein levels.

### Example 2: Characterisation of RetGC KO Organoids

RetGC KO and WT retinal organoids were generated from human induced pluripotent cells (hiPSCs) using an established differentiation protocol. The differentiation protocol produced retinal organoids that are ‘mature’ at day 140 (20 weeks) and could be used in AAV transduction experiments. Mature retinal organoids could be maintained in culture for up to day 300 (43 weeks) without morphologically distinguishable signs of degeneration.

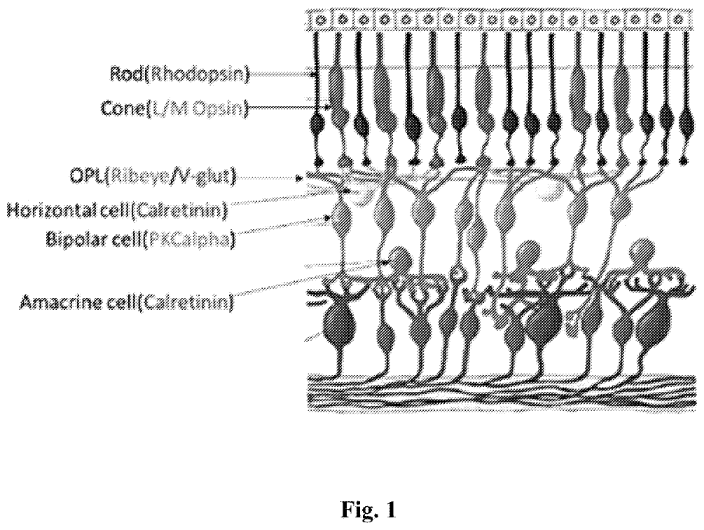

The human neural retina is structured in several layers of nerve cells including horizontal cells, bipolar cells, amacrine cells, muller glia and ganglion cells, photoreceptors, retinal pigment epithelial cells (FIG. 1). The in vitro generated organoids reflect the laminated morphology of the neural retina with the above retinal cell types arranged in their appropriate layers and connected in two synaptic layers.

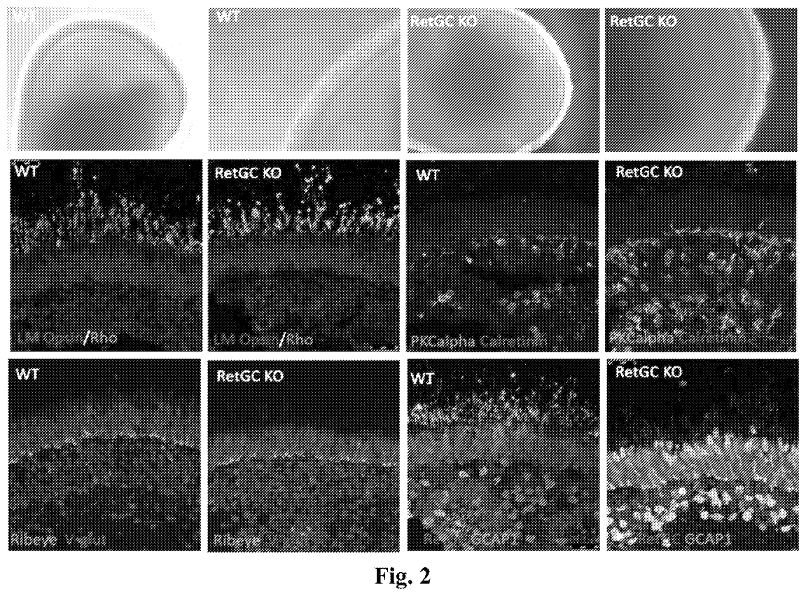

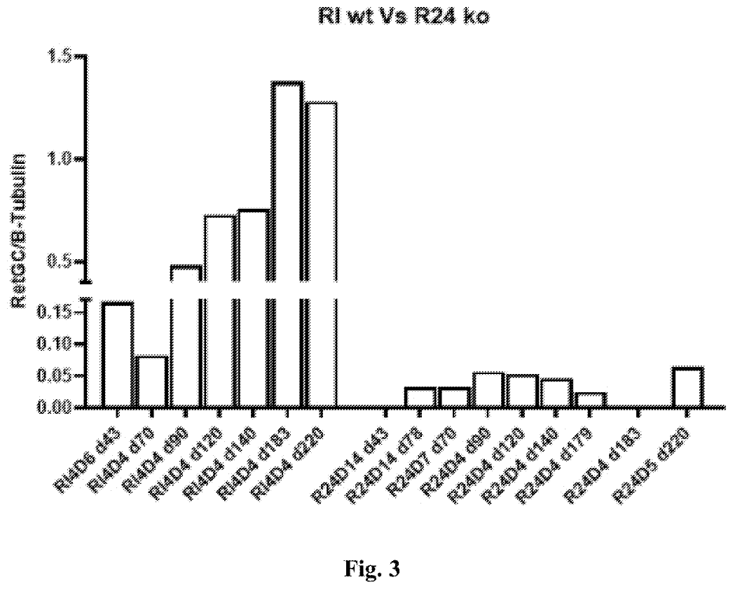

The WT and RetGC KO organoids were characterized using immunofluorescence, Western blot and qPCR techniques. The relevant markers for the different cell types in the retina were used to identify and illustrate the similarity in retinal morphology between in vivo human retina and retinal organoids in both the WT and RetGC KO cell lines. FIG. 2 shows cryosectioned and immuno-stained images of LM Opsin and Rhodopsin for cone and rod photoreceptors, Ribeye and V Glut for synapses in the outer plexiform layer, PKCa and Calretinin for the bipolar, horizontal and amacrine cells. The brightfield images depict mature organoids with visible ‘brush borders’ which are the photoreceptor outer segments. The graph in FIG. 3 shows analysis of RetGC protein expression over the time course of retinal organoid development (day 40 to day 220). RetGC protein levels are significantly reduced in RetGC KO organoids relative to WT.

### Example 3: Design of Vectors to Restore RetGC Expression in KO Organoids

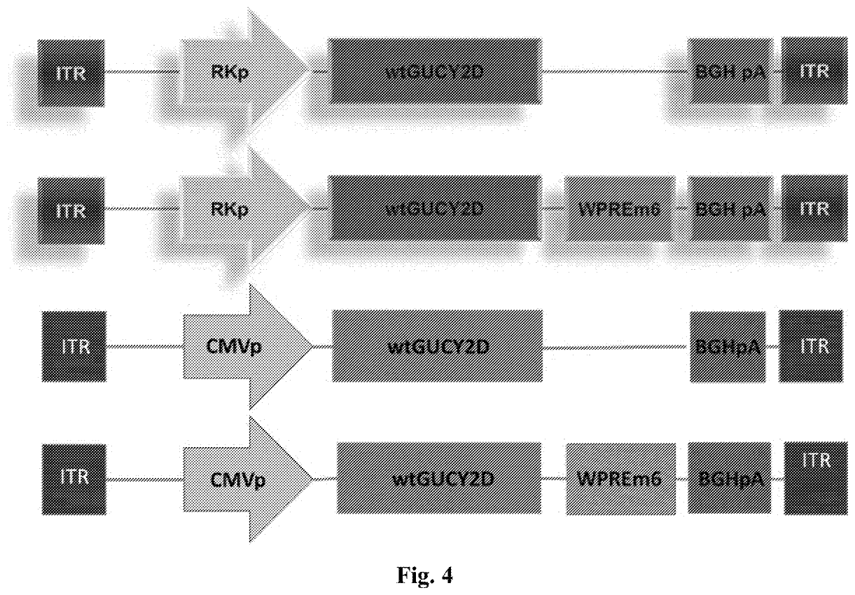

Viral vectors comprising one of four different expression constructs were designed as shown in FIG. 4. The expression constructs had two different promoters: RK (derived from the photoreceptor specific rhodopsin kinase promoter specific to photoreceptors) and CMV
(derived from cytomegalovirus). Some of the expression constructs also contained a woodchuck hepatitis virus post transcriptional regulatory element (WPRE). All viral genomes were packaged into 7m8 capsid.

The WT and RetGC KO retinal organoids were transduced at an age ranging from day 140 to day 204 with the four different viral vectors and incubated for 21 days before harvesting and analysis. The transduced organoids were assessed using immunofluorescence, Western blotting, qPCR, and cGMP FRET assay.

All four AAV 7m8 vectors successfully transduced human photoreceptors and driving RetGC protein expression in RetGC KO retinal organoids as determined by total RetGC protein quantification (Western blot) and mRNA (qPCR). Transgenic RetGC delivered by 7m8 CMV-RetGC and 7m8 RK-RetGC was detectable by immunofluorescence in the correct intracellular compartment of the photoreceptor outer segment.

### Example 4: AAV Vector Driven RetGC Expression Restores PDE6β Expression in Photoreceptor Outer Segments

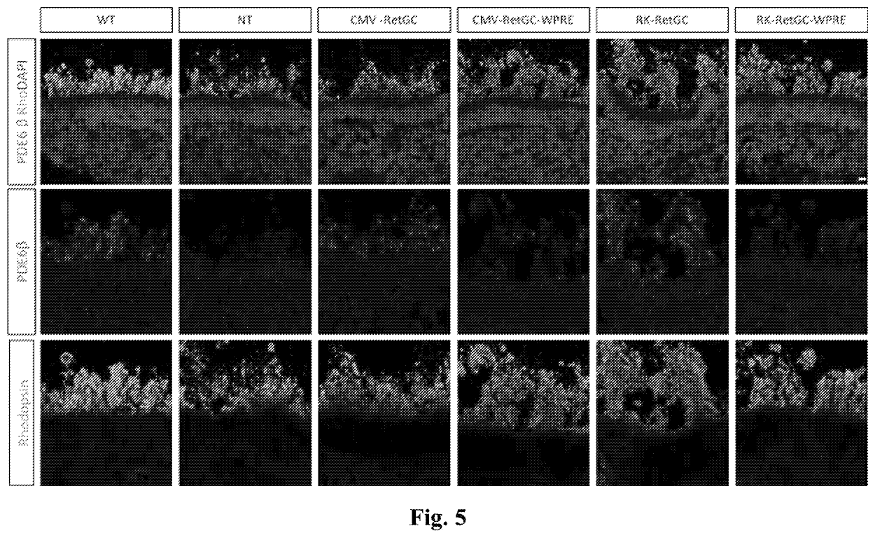

FIG. 5 shows the immunostaining of PDE6β in WT, non-transduced and viral vector transduced retinal organoids. PDE6β was co-stained with Rhodopsin protein to establish the presence of outer segments in all organoids and depict how reduced the PDE6β protein was in the non-transduced control compared to the WT control. After transduction with the viral vectors, the restoration of PDE6β protein was verified.

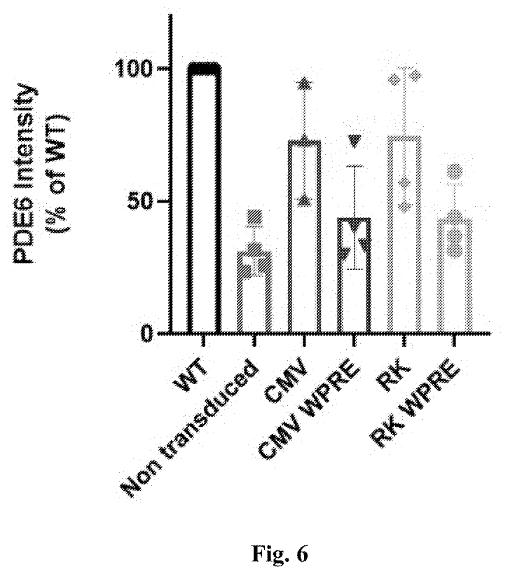

There was significant reduction in PDE6β staining intensity non-transduced RetGC KO relative to WT control retinal organoids, p<0.005 (a one way ANOVA test was applied with Kruskal-Wallis test for multiple comparisons). Staining intensity in rhodopsin positive outer segments was quantified in multiple WT, RetGC KO and transduced organoids. PDE6β expression was restored close to WT levels in organoids which had been treated with 7m8-CMV-RetGC and 7m8-RK-RetGC. 7m8-CMV-RetGC-WPRE and 7m8-RK-RetGC-WPRE showed improvement compared to KO, but not to the same level as other two vectors (FIG. 6 and Table 1).

TABLE 1
Restoration of PDE6β expression in outer segments.

| | PDE6β compared | PDE6β compared |
|---|---|---|
| Vector | to WT [%] | to WT [SD, %] |
| 7m8-CMV-RetGC | 73 | 22.0 |
| 7m8-RK-RetGC | 75 | 25.7 |
| 7m8-CMV-RetGC-WPRE | 44 | 19.5 |
| 7m8-RK-RetGC-WPRE | 43 | 13.0 |

SD = standard deviation.
n = 4 for each vector.

### Example 5: AAV Vector Driven RetGC Expression Restores RetGC Protein Levels

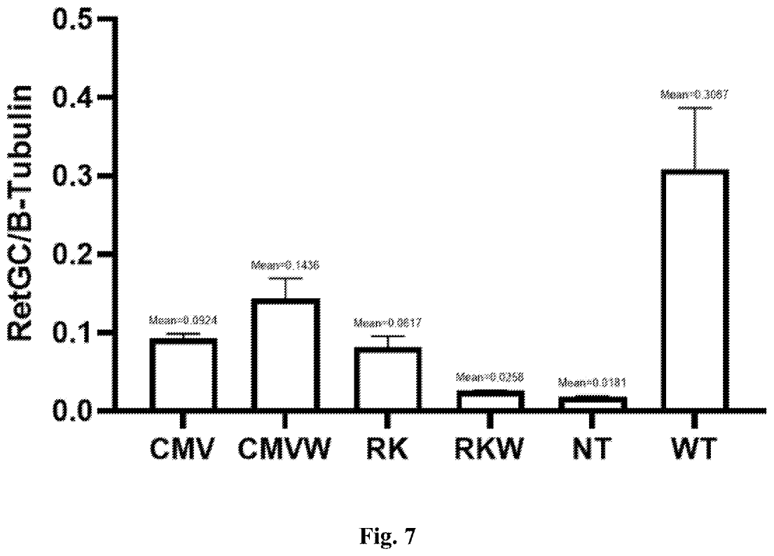

RetGC protein levels were assayed by Western Blot. As shown in FIG. 7, RetGC expression was higher in the EBs transduced with the vectors 7m8-CMV-RetGC (30% of WT), 7m8-CMV-WPRE-RetGC (47% of WT) and 7m8-RK-RetGC (27% of WT) with respect the non-transduced EBs. For each experimental group two samples were harvested and processed for protein expression analysis.

### Example 6: AAV Vector Driven RetGC Expression Restores Total cGMP Levels in Organoids Following Light Stimulation

To measure RetGC activity, the quantitative measurement of cGMP was carried out in a competitive assay format using a specific antibody labelled with Europium Cryptate (donor) and cGMP labelled with d2 Reagent (acceptor). The detection principle is based on HTRF® technology. When the dyes are in close proximity, the excitation of the donor with a light source (laser or flash lamp) triggers a Fluorescence Resonance Energy Transfer (FRET) towards the acceptor, which in turn fluoresces at a specific wavelength (665 nm). The cGMP present in the sample competes with the binding between the two conjugates and thereby prevents FRET from occurring. The specific signal is inversely proportional to the cGMP concentration.

WT and KO organoids, transduced and non-transduced with the 7m8 vectors, were exposed to a cycle of light/dark to induce the production of cGMP. The light stimulation protocol used, consisted of 5 min of white light stimulation and 5 min of dark before the dissection of the organoids to isolate the photoreceptors. The samples were dissected and lysed under red light in the presence of IBMX (PDE-inhibitor) as described in the study protocol. The assay determines cGMP [nM] concentration relative to a standard curve and the values obtained were normalised on the total protein amount [ug] per sample. The statistical analysis was performed to evaluate statistical difference between the samples compared to the Non-transduced KO control (NT).

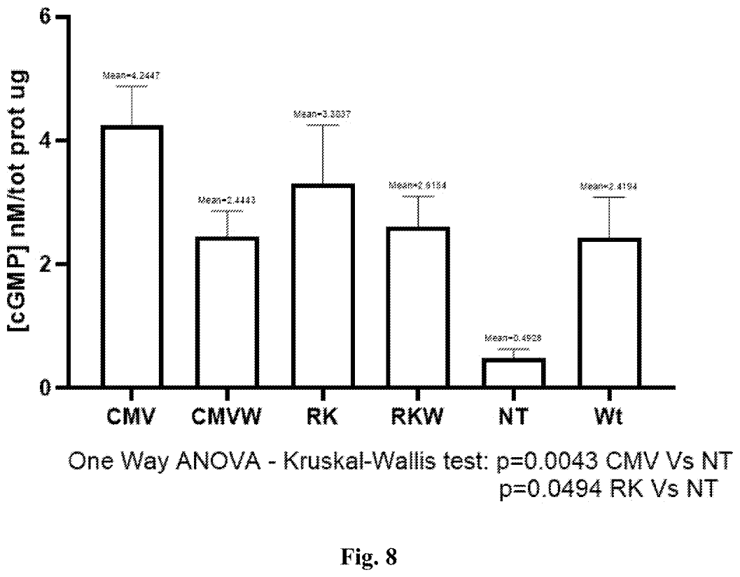

As is shown in the graph in the FIG. 8, RetGC KO organoids (NT) had a significant reduction in cGMP levels post light stimulation (20% of WT). Following transduction, a statistically significant increase in cGMP was found in the KO RetGC-GUCY2D organoids transduced with the vectors 7m8-CMV-GUCY2D (+76% of WT, p=0.0043) and 7m8-RK-GUCY2D (+37% of WT, p=0.0494). Transductions with both the CMV and RK vectors carrying the WPRE element led to an increase in cGMP that was not statistically significant but with a mean value comparable to the one found in the WT samples. The graph shows the results obtained from two separate experiments with 3 or 4 transduced organoids per group (FIG. 8). The observation that total cGMP levels met and exceeded WT levels demonstrate the functional potency of these above cited vectors in the context of light sensitive human photoreceptors.

Overview of sequences

| SEQ | | | |
|---|---|---|---|
| ID | | | |
| NO | Name | Description | Sequence |
| 1 | AAVss- | AAV2 5′ ITR: | CCTGCAGGCAGCTGCGCGCTCGCTCGCTCACTGA |
| | RK- | 1-141 bp | GGCCGCCCGGGCAAAGCCCGGGCGTCGGGCGACC |
| | hGUCY2D- | RK: 169-620 bp | TTTGGTCGCCCGGCCTCAGTGAGCGAGCGAGCGC |
| | WPRE(mut6) | Kozak: 813-818 | GCAGAGAGGGAGTGGCCAACTCCATCACTAGGGG |
| | | bp | TTCCTTCTAGACAACTTTGTATAGAAAAGTTGTGT |
| | | hGUCY2D [cds | AGTTAATGATTAACCCGCCATGCTACTTATCTACG |
| | | from | TACATTTATATTGGCTCATGTCCAACATTACCGCC |
| | | NM_000180.4]: | ATGTTGACATTGATTATTGACTAGAATTCGCTAGC |
| | | 819-4130 bp | AAGATCCAAGCTCAGATCTCGATCGAGTTGGGCC |
| | | WPREmut6: | CCAGAAGCCTGGTGGTTGTTTGTCCTTCTCAGGGG |
| | | 4131-4719 bp | AAAAGTGAGGCGGCCCCTTGGAGGAAGGGGCCG |
| | | BGH pA: 4774- | GGCAGAATGATCTAATCGGATTCCAAGCAGCTCA |
| | | 4981 bp | GGGGATTGTCTTTTTCTAGCACCTTCTTGCCACTC |
| | | AAV2 3′ ITR: | CTAAGCGTCCTCCGTGACCCCGGCTGGGATTTAGC |
| | | 4989-5129 bp | CTGGTGCTGTGTCAGCCCCGGTCTCCCAGGGGCTT |
| | | | CCCAGTGGTCCCCAGGAACCCTCGACAGGGCCCG |
| | | | GTCTCTCTCGTCCAGCAAGGGCAGGGACGGGCCA |
| | | | CAGGCCAAGGGCCCTCGATCGAGGAACTGAAAAA |
| | | | CCAGAAAGTTAACTGGTAAGTTTAGTCTTTTTGTC |
| | | | TTTTATTTCAGGTCCCGGATCCGGTGGTGGTGCAA |
| | | | ATCAAAGAACTGCTCCTCAGTGGATGTTGCCTTTA |
| | | | CTTCTAGGCCTGTACGGAAGTGTTACTTCTGCTCT |
| | | | AAAAGCTGCGGAATTGTACCCGCGGCCGCCAAGT |
| | | | TTGTACAAAAAAGCAGGCTGCCACCATGACCGCC |
| | | | TGCGCCCGCCGAGCGGGTGGGCTTCCGGACCCCG |
| | | | GGCTCTGCGGTCCCGCGTGGTGGGCTCCGTCCCTG |
| | | | CCCCGCCTCCCCCGGGCCCTGCCCCGGCTCCCGCT |
| | | | CCTGCTGCTCCTGCTTCTGCTGCAGCCCCCCGCCC |
| | | | TCTCCGCCGTGTTCACGGTGGGGGTCCTGGGCCCC |
| | | | TGGGCTTGCGACCCCATCTTCTCTCGGGCTCGCCC |
| | | | GGACCTGGCCGCCCGCCTGGCCGCCGCCCGCCTG |
| | | | AACCGCGACCCCGGCCTGGCAGGCGGTCCCCGCT |
| | | | TCGAGGTAGCGCTGCTGCCCGAGCCTTGCCGGAC |
| | | | GCCGGGCTCGCTGGGGGCCGTGTCCTCCGCGCTG |
| | | | GCCCGCGTGTCGGGCCTCGTGGGTCCGGTGAACC |
| | | | CTGCGGCCTGCCGGCCAGCCGAGCTGCTCGCCGA |
| | | | AGAAGCCGGGATCGCGCTGGTGCCCTGGGGCTGC |
| | | | CCCTGGACGCAGGCGGAGGGCACCACGGCCCCTG |
| | | | CCGTGACCCCCGCCGCGGATGCCCTCTACGCCCTG |
| | | | CTTCGCGCATTCGGCTGGGCGCGCGTGGCCCTGGT |
| | | | CACCGCCCCCCAGGACCTGTGGGTGGAGGCGGGA |
| | | | CGCTCACTGTCCACGGCACTCAGGGCCCGGGGCC |
| | | | TGCCTGTCGCCTCCGTGACTTCCATGGAGCCCTTG |
| | | | GACCTGTCTGGAGCCCGGGAGGCCCTGAGGAAGG |
| | | | TTCGGGACGGGCCCAGGGTCACAGCAGTGATCAT |
| | | | GGTGATGCACTCGGTGCTGCTGGGTGGCGAGGAG |
| | | | CAGCGCTACCTCCTGGAGGCCGCAGAGGAGCTGG |
| | | | GCCTGACCGATGGCTCCCTGGTCTTCCTGCCCTTC |
| | | | GACACGATCCACTACGCCTTGTCCCCAGGCCCGG |
| | | | AGGCCTTGGCCGCACTCGCCAACAGCTCCCAGCT |
| | | | TCGCAGGGCCCACGATGCCGTGCTCACCCTCACG |
| | | | CGCCACTGTCCCTCTGAAGGCAGCGTGCTGGACA |
| | | | GCCTGCGCAGGGCTCAAGAGCGCCGCGAGCTGCC |
| | | | CTCTGACCTCAATCTGCAGCAGGTCTCCCCACTCT |
| | | | TTGGCACCATCTATGACGCGGTCTTCTTGCTGGCA |
| | | | AGGGGCGTGGCAGAAGCGCGGGCTGCCGCAGGT |
| | | | GGCAGATGGGTGTCCGGAGCAGCTGTGGCCCGCC |
| | | | ACATCCGGGATGCGCAGGTCCCTGGCTTCTGCGG |
| | | | GGACCTAGGAGGAGACGAGGAGCCCCCATTCGTG |
| | | | CTGCTAGACACGGACGCGGCGGGAGACCGGCTTT |
| | | | TTGCCACATACATGCTGGATCCTGCCCGGGGCTCC |
| | | | TTCCTCTCCGCCGGTACCCGGATGCACTTCCCGCG |
| | | | TGGGGGATCAGCACCCGGACCTGACCCCTCGTGC |
| | | | TGGTTCGATCCAAACAACATCTGCGGTGGAGGAC |
| | | | TGGAGCCGGGCCTCGTCTTTCTTGGCTTCCTCCTG |
| | | | GTGGTTGGGATGGGGCTGGCTGGGGCCTTCCTGG |
| | | | CCCATTATGTGAGGCACCGGCTACTTCACATGCA |
| | | | AATGGTCTCCGGCCCCAACAAGATCATCCTGACC |
| | | | GTGGACGACATCACCTTTCTCCACCCACATGGGG |
| | | | GCACCTCTCGAAAGGTGGCCCAGGGGAGTCGATC |
| | | | AAGTCTGGGTGCCCGCAGCATGTCAGACATTCGC |
| | | | AGCGGCCCCAGCCAACACTTGGACAGCCCCAACA |
| | | | TTGGTGTCTATGAGGGAGACAGGGTTTGGCTGAA |
| | | | GAAATTCCCAGGGGATCAGCACATAGCTATCCGC |
| | | | CCAGCAACCAAGACGGCCTTCTCCAAGCTCCAGG |
| | | | AGCTCCGGCATGAGAACGTGGCCCTCTACCTGGG |
| | | | GCTTTTCCTGGCTCGGGGAGCAGAAGGCCCTGCG |
| | | | GCCCTCTGGGAGGGCAACCTGGCTGTGGTCTCAG |
| | | | AGCACTGCACGCGGGGCTCTCTTCAGGACCTCCTC |
| | | | GCTCAGAGAGAAATAAAGCTGGACTGGATGTTCA |
| | | | AGTCCTCCCTCCTGCTGGACCTTATCAAGGGAATA |
| | | | AGGTATCTGCACCATCGAGGCGTGGCTCATGGGC |
| | | | GGCTGAAGTCACGGAACTGCATAGTGGATGGCAG |
| | | | ATTCGTACTCAAGATCACTGACCACGGCCACGGG |
| | | | AGACTGCTGGAAGCACAGAAGGTGCTACCGGAGC |
| | | | CTCCCAGAGCGGAGGACCAGCTGTGGACAGCCCC |
| | | | GGAGCTGCTTAGGGACCCAGCCCTGGAGCGCCGG |
| | | | GGAACGCTGGCCGGCGACGTCTTTAGCTTGGCCA |
| | | | TCATCATGCAAGAAGTAGTGTGCCGCAGTGCCCC |
| | | | TTATGCCATGCTGGAGCTCACTCCCGAGGAAGTG |
| | | | GTGCAGAGGGTGCGGAGCCCCCCTCCACTGTGTC |
| | | | GGCCCTTGGTGTCCATGGACCAGGCACCTGTCGA |
| | | | GTGTATCCTCCTGATGAAGCAGTGCTGGGCAGAG |
| | | | CAGCCGGAACTTCGGCCCTCCATGGACCACACCT |
| | | | TCGACCTGTTCAAGAACATCAACAAGGGCCGGAA |
| | | | GACGAACATCATTGACTCGATGCTTCGGATGCTG |
| | | | GAGCAGTACTCTAGTAACCTGGAGGATCTGATCC |
| | | | GGGAGCGCACGGAGGAGCTGGAGCTGGAAAAGC |
| | | | AGAAGACAGACCGGCTGCTTACACAGATGCTGCC |
| | | | TCCGTCTGTGGCTGAGGCCTTGAAGACGGGGACA |
| | | | CCAGTGGAGCCCGAGTACTTTGAGCAAGTGACAC |
| | | | TGTACTTTAGTGACATTGTGGGCTTCACCACCATC |
| | | | TCTGCCATGAGTGAGCCCATTGAGGTTGTGGACCT |
| | | | GCTCAACGATCTCTACACACTCTTTGATGCCATCA |
| | | | TTGGTTCCCACGATGTCTACAAGGTGGAGACAAT |
| | | | AGGGGACGCCTATATGGTGGCCTCGGGGCTGCCC |
| | | | CAGCGGAATGGGCAGCGACACGCGGCAGAGATC |
| | | | GCCAACATGTCACTGGACATCCTCAGTGCCGTGG |
| | | | GCACTTTCCGCATGCGCCATATGCCTGAGGTTCCC |
| | | | GTGCGCATCCGCATAGGCCTGCACTCGGGTCCAT |
| | | | GCGTGGCAGGCGTGGTGGGCCTCACCATGCCGCG |
| | | | GTACTGCCTGTTTGGGGACACGGTCAACACCGCC |
| | | | TCGCGCATGGAGTCCACCGGGCTGCCTTACCGCA |
| | | | TCCACGTGAACTTGAGCACTGTGGGGATTCTCCGT |
| | | | GCTCTGGACTCGGGCTACCAGGTGGAGCTGCGAG |
| | | | GCCGCACGGAGCTGAAGGGCAAGGGCGCCGAGG |
| | | | ACACTTTCTGGCTAGTGGGCAGACGCGGCTTCAA |
| | | | CAAGCCCATCCCCAAACCGCCTGACCTGCAACCG |
| | | | GGGTCCAGCAACCACGGCATCAGCCTGCAGGAGA |
| | | | TCCCACCCGAGCGGCGACGGAAGCTGGAGAAGGC |
| | | | GCGGCCGGGCCAGTTCTCTTGAAATCAACCTCTG |
| | | | GATTACAAAATTTGTGAAAGATTGACTGGTATTCT |
| | | | TAACTATGTTGCTCCTTTTACGCTATGTGGATACG |
| | | | CTGCTTTAATGCCTTTGTATCATGCTATTGCTTCCC |
| | | | GTATGGCTTTCATTTTCTCCTCCTTGTATAAATCCT |
| | | | GGTTGCTGTCTCTTTATGAGGAGTTGTGGCCCGTT |
| | | | GTCAGGCAACGTGGCGTGGTGTGCACTGTGTTTG |
| | | | CTGACGCAACCCCCACTGGTTGGGGCATTGCCAC |
| | | | CACCTGTCAGCTCCTTTCCGGGACTTTCGCTTTCC |
| | | | CCCTCCCTATTGCCACGGCGGAACTCATCGCCGCC |
| | | | TGCCTTGCCCGCTGCTGGACAGGGGCTCGGCTGTT |
| | | | GGGCACTGACAATTCCGTGGTGTTGTCGGGGAAA |
| | | | TCATCGTCCTTTCCTTGGCTGCTCGCCTGTGTTGCC |
| | | | ACCTGGATTCTGCGCGGGACGTCCTTCTGCTACGT |
| | | | CCCTTCGGCCCTCAATCCAGCGGACCTTCCTTCCC |
| | | | GCGGCCTGCTGCCGGCTCTGCGGCCTCTTCCGCGT |
| | | | CTTCGCCTTCGCCCTCAGACGAGTCGGATCTCCCT |
| | | | TTGGGCCGCCTCCCCGCACCCAGCTTTCTTGTACA |
| | | | AAGTGGGAATTCCTAGAGCTCGCTGATCAGCCTC |
| | | | GACTGTGCCTTCTAGTTGCCAGCCATCTGTTGTTT |
| | | | GCCCCTCCCCCGTGCCTTCCTTGACCCTGGAAGGT |
| | | | GCCACTCCCACTGTCCTTTCCTAATAAAATGAGGA |
| | | | AATTGCATCGCATTGTCTGAGTAGGTGTCATTCTA |
| | | | TTCTGGGGGGTGGGGTGGGGCAGGACAGCAAGG |
| | | | GGGAGGATTGGGAAGAGAATAGCAGGCATGCTG |
| | | | GGGAGGGCCGCAGGAACCCCTAGTGATGGAGTTG |
| | | | GCCACTCCCTCTCTGCGCGCTCGCTCGCTCACTGA |
| | | | GGCCGGGCGACCAAAGGTCGCCCGACGCCCGGGC |
| | | | TTTGCCCGGGCGGCCTCAGTGAGCGAGCGAGCGC |
| | | | GCAGCTGCCTGCAGG |
| 2 | AAVss- | AAV2 5′ ITR: | CCTGCAGGCAGCTGCGCGCTCGCTCGCTCACTGA |
| | RK- | 1-141 bp | GGCCGCCCGGGCAAAGCCCGGGCGTCGGGCGACC |
| | GUCY2D | RK: 169-620 bp | TTTGGTCGCCCGGCCTCAGTGAGCGAGCGAGCGC |
| | | Kozak: 813-818 | GCAGAGAGGGAGTGGCCAACTCCATCACTAGGGG |
| | | bp | TTCCTTCTAGACAACTTTGTATAGAAAAGTTGTGT |
| | | hGUCY2D [cds | AGTTAATGATTAACCCGCCATGCTACTTATCTACG |
| | | from | TACATTTATATTGGCTCATGTCCAACATTACCGCC |
| | | NM_000180.4]: | ATGTTGACATTGATTATTGACTAGAATTCGCTAGC |
| | | 819-4130 bp | AAGATCCAAGCTCAGATCTCGATCGAGTTGGGCC |
| | | BGH pA: 4185- | CCAGAAGCCTGGTGGTTGTTTGTCCTTCTCAGGGG |
| | | 4392 bp | AAAAGTGAGGCGGCCCCTTGGAGGAAGGGGCCG |
| | | AAV2 3′ ITR: | GGCAGAATGATCTAATCGGATTCCAAGCAGCTCA |
| | | 4400-4540 bp | GGGGATTGTCTTTTTCTAGCACCTTCTTGCCACTC |
| | | | CTAAGCGTCCTCCGTGACCCCGGCTGGGATTTAGC |
| | | | CTGGTGCTGTGTCAGCCCCGGTCTCCCAGGGGCTT |
| | | | CCCAGTGGTCCCCAGGAACCCTCGACAGGGCCCG |
| | | | GTCTCTCTCGTCCAGCAAGGGCAGGGACGGGCCA |
| | | | CAGGCCAAGGGCCCTCGATCGAGGAACTGAAAAA |
| | | | CCAGAAAGTTAACTGGTAAGTTTAGTCTTTTTGTC |
| | | | TTTTATTTCAGGTCCCGGATCCGGTGGTGGTGCAA |
| | | | ATCAAAGAACTGCTCCTCAGTGGATGTTGCCTTTA |
| | | | CTTCTAGGCCTGTACGGAAGTGTTACTTCTGCTCT |
| | | | AAAAGCTGCGGAATTGTACCCGCGGCCGCCAAGT |
| | | | TTGTACAAAAAAGCAGGCTGCCACCATGACCGCC |
| | | | TGCGCCCGCCGAGCGGGTGGGCTTCCGGACCCCG |
| | | | GGCTCTGCGGTCCCGCGTGGTGGGCTCCGTCCCTG |
| | | | CCCCGCCTCCCCCGGGCCCTGCCCCGGCTCCCGCT |
| | | | CCTGCTGCTCCTGCTTCTGCTGCAGCCCCCCGCCC |
| | | | TCTCCGCCGTGTTCACGGTGGGGGTCCTGGGCCCC |
| | | | TGGGCTTGCGACCCCATCTTCTCTCGGGCTCGCCC |
| | | | GGACCTGGCCGCCCGCCTGGCCGCCGCCCGCCTG |
| | | | AACCGCGACCCCGGCCTGGCAGGCGGTCCCCGCT |
| | | | TCGAGGTAGCGCTGCTGCCCGAGCCTTGCCGGAC |
| | | | GCCGGGCTCGCTGGGGGCCGTGTCCTCCGCGCTG |
| | | | GCCCGCGTGTCGGGCCTCGTGGGTCCGGTGAACC |
| | | | CTGCGGCCTGCCGGCCAGCCGAGCTGCTCGCCGA |
| | | | AGAAGCCGGGATCGCGCTGGTGCCCTGGGGCTGC |
| | | | CCCTGGACGCAGGCGGAGGGCACCACGGCCCCTG |
| | | | CCGTGACCCCCGCCGCGGATGCCCTCTACGCCCTG |
| | | | CTTCGCGCATTCGGCTGGGCGCGCGTGGCCCTGGT |
| | | | CACCGCCCCCCAGGACCTGTGGGTGGAGGCGGGA |
| | | | CGCTCACTGTCCACGGCACTCAGGGCCCGGGGCC |
| | | | TGCCTGTCGCCTCCGTGACTTCCATGGAGCCCTTG |
| | | | GACCTGTCTGGAGCCCGGGAGGCCCTGAGGAAGG |
| | | | TTCGGGACGGGCCCAGGGTCACAGCAGTGATCAT |
| | | | GGTGATGCACTCGGTGCTGCTGGGTGGCGAGGAG |
| | | | CAGCGCTACCTCCTGGAGGCCGCAGAGGAGCTGG |
| | | | GCCTGACCGATGGCTCCCTGGTCTTCCTGCCCTTC |
| | | | GACACGATCCACTACGCCTTGTCCCCAGGCCCGG |
| | | | AGGCCTTGGCCGCACTCGCCAACAGCTCCCAGCT |
| | | | TCGCAGGGCCCACGATGCCGTGCTCACCCTCACG |
| | | | CGCCACTGTCCCTCTGAAGGCAGCGTGCTGGACA |
| | | | GCCTGCGCAGGGCTCAAGAGCGCCGCGAGCTGCC |
| | | | CTCTGACCTCAATCTGCAGCAGGTCTCCCCACTCT |
| | | | TTGGCACCATCTATGACGCGGTCTTCTTGCTGGCA |
| | | | AGGGGCGTGGCAGAAGCGCGGGCTGCCGCAGGT |
| | | | GGCAGATGGGTGTCCGGAGCAGCTGTGGCCCGCC |
| | | | ACATCCGGGATGCGCAGGTCCCTGGCTTCTGCGG |
| | | | GGACCTAGGAGGAGACGAGGAGCCCCCATTCGTG |
| | | | CTGCTAGACACGGACGCGGCGGGAGACCGGCTTT |
| | | | TTGCCACATACATGCTGGATCCTGCCCGGGGCTCC |
| | | | TTCCTCTCCGCCGGTACCCGGATGCACTTCCCGCG |
| | | | TGGGGGATCAGCACCCGGACCTGACCCCTCGTGC |
| | | | TGGTTCGATCCAAACAACATCTGCGGTGGAGGAC |
| | | | TGGAGCCGGGCCTCGTCTTTCTTGGCTTCCTCCTG |
| | | | GTGGTTGGGATGGGGCTGGCTGGGGCCTTCCTGG |
| | | | CCCATTATGTGAGGCACCGGCTACTTCACATGCA |
| | | | AATGGTCTCCGGCCCCAACAAGATCATCCTGACC |
| | | | GTGGACGACATCACCTTTCTCCACCCACATGGGG |
| | | | GCACCTCTCGAAAGGTGGCCCAGGGGAGTCGATC |
| | | | AAGTCTGGGTGCCCGCAGCATGTCAGACATTCGC |
| | | | AGCGGCCCCAGCCAACACTTGGACAGCCCCAACA |
| | | | TTGGTGTCTATGAGGGAGACAGGGTTTGGCTGAA |
| | | | GAAATTCCCAGGGGATCAGCACATAGCTATCCGC |
| | | | CCAGCAACCAAGACGGCCTTCTCCAAGCTCCAGG |
| | | | AGCTCCGGCATGAGAACGTGGCCCTCTACCTGGG |
| | | | GCTTTTCCTGGCTCGGGGAGCAGAAGGCCCTGCG |
| | | | GCCCTCTGGGAGGGCAACCTGGCTGTGGTCTCAG |
| | | | AGCACTGCACGCGGGGCTCTCTTCAGGACCTCCTC |
| | | | GCTCAGAGAGAAATAAAGCTGGACTGGATGTTCA |
| | | | AGTCCTCCCTCCTGCTGGACCTTATCAAGGGAATA |
| | | | AGGTATCTGCACCATCGAGGCGTGGCTCATGGGC |
| | | | GGCTGAAGTCACGGAACTGCATAGTGGATGGCAG |
| | | | ATTCGTACTCAAGATCACTGACCACGGCCACGGG |
| | | | AGACTGCTGGAAGCACAGAAGGTGCTACCGGAGC |
| | | | CTCCCAGAGCGGAGGACCAGCTGTGGACAGCCCC |
| | | | GGAGCTGCTTAGGGACCCAGCCCTGGAGCGCCGG |
| | | | GGAACGCTGGCCGGCGACGTCTTTAGCTTGGCCA |
| | | | TCATCATGCAAGAAGTAGTGTGCCGCAGTGCCCC |
| | | | TTATGCCATGCTGGAGCTCACTCCCGAGGAAGTG |
| | | | GTGCAGAGGGTGCGGAGCCCCCCTCCACTGTGTC |
| | | | GGCCCTTGGTGTCCATGGACCAGGCACCTGTCGA |
| | | | GTGTATCCTCCTGATGAAGCAGTGCTGGGCAGAG |
| | | | CAGCCGGAACTTCGGCCCTCCATGGACCACACCT |
| | | | TCGACCTGTTCAAGAACATCAACAAGGGCCGGAA |
| | | | GACGAACATCATTGACTCGATGCTTCGGATGCTG |
| | | | GAGCAGTACTCTAGTAACCTGGAGGATCTGATCC |
| | | | GGGAGCGCACGGAGGAGCTGGAGCTGGAAAAGC |
| | | | AGAAGACAGACCGGCTGCTTACACAGATGCTGCC |
| | | | TCCGTCTGTGGCTGAGGCCTTGAAGACGGGGACA |
| | | | CCAGTGGAGCCCGAGTACTTTGAGCAAGTGACAC |
| | | | TGTACTTTAGTGACATTGTGGGCTTCACCACCATC |
| | | | TCTGCCATGAGTGAGCCCATTGAGGTTGTGGACCT |
| | | | GCTCAACGATCTCTACACACTCTTTGATGCCATCA |
| | | | TTGGTTCCCACGATGTCTACAAGGTGGAGACAAT |
| | | | AGGGGACGCCTATATGGTGGCCTCGGGGCTGCCC |
| | | | CAGCGGAATGGGCAGCGACACGCGGCAGAGATC |
| | | | GCCAACATGTCACTGGACATCCTCAGTGCCGTGG |
| | | | GCACTTTCCGCATGCGCCATATGCCTGAGGTTCCC |
| | | | GTGCGCATCCGCATAGGCCTGCACTCGGGTCCAT |
| | | | GCGTGGCAGGCGTGGTGGGCCTCACCATGCCGCG |
| | | | GTACTGCCTGTTTGGGGACACGGTCAACACCGCC |
| | | | TCGCGCATGGAGTCCACCGGGCTGCCTTACCGCA |
| | | | TCCACGTGAACTTGAGCACTGTGGGGATTCTCCGT |
| | | | GCTCTGGACTCGGGCTACCAGGTGGAGCTGCGAG |
| | | | GCCGCACGGAGCTGAAGGGCAAGGGCGCCGAGG |
| | | | ACACTTTCTGGCTAGTGGGCAGACGCGGCTTCAA |
| | | | CAAGCCCATCCCCAAACCGCCTGACCTGCAACCG |
| | | | GGGTCCAGCAACCACGGCATCAGCCTGCAGGAGA |
| | | | TCCCACCCGAGCGGCGACGGAAGCTGGAGAAGGC |
| | | | GCGGCCGGGCCAGTTCTCTTGAACCCAGCTTTCTT |
| | | | GTACAAAGTGGGAATTCCTAGAGCTCGCTGATCA |
| | | | GCCTCGACTGTGCCTTCTAGTTGCCAGCCATCTGT |
| | | | TGTTTGCCCCTCCCCCGTGCCTTCCTTGACCCTGG |
| | | | AAGGTGCCACTCCCACTGTCCTTTCCTAATAAAAT |
| | | | GAGGAAATTGCATCGCATTGTCTGAGTAGGTGTC |
| | | | ATTCTATTCTGGGGGGTGGGGTGGGGCAGGACAG |
| | | | CAAGGGGGAGGATTGGGAAGAGAATAGCAGGCA |
| | | | TGCTGGGGAGGGCCGCAGGAACCCCTAGTGATGG |
| | | | AGTTGGCCACTCCCTCTCTGCGCGCTCGCTCGCTC |
| | | | ACTGAGGCCGGGCGACCAAAGGTCGCCCGACGCC |
| | | | CGGGCTTTGCCCGGGCGGCCTCAGTGAGCGAGCG |
| | | | AGCGCGCAGCTGCCTGCAGG |
| 3 | AAVss- | AAV2 5′ ITR: | CCTGCAGGCAGCTGCGCGCTCGCTCGCTCACTGA |
| | CMV- | 1-141 bp | GGCCGCCCGGGCAAAGCCCGGGCGTCGGGCGACC |
| | hGUCY2D- | CMV: 169-757 | TTTGGTCGCCCGGCCTCAGTGAGCGAGCGAGCGC |
| | WPRE(mut6) | bp | GCAGAGAGGGAGTGGCCAACTCCATCACTAGGGG |
| | | Kozak: 782-787 | TTCCTTCTAGACAACTTTGTATAGAAAAGTTGTAG |
| | | bp | TTATTAATAGTAATCAATTACGGGGTCATTAGTTC |
| | | hGUCY2D [cds | ATAGCCCATATATGGAGTTCCGCGTTACATAACTT |
| | | from | ACGGTAAATGGCCCGCCTGGCTGACCGCCCAACG |
| | | NM_000180.4]: | ACCCCCGCCCATTGACGTCAATAATGACGTATGTT |
| | | 788-4099 bp | CCCATAGTAACGCCAATAGGGACTTTCCATTGAC |
| | | WPRE(mut6): | GTCAATGGGTGGAGTATTTACGGTAAACTGCCCA |
| | | 4100-4688 bp | CTTGGCAGTACATCAAGTGTATCATATGCCAAGT |
| | | BGH pA: 4743- | ACGCCCCCTATTGACGTCAATGACGGTAAATGGC |
| | | 4950 bp | CCGCCTGGCATTATGCCCAGTACATGACCTTATGG |
| | | AAV2 3′ ITR: | GACTTTCCTACTTGGCAGTACATCTACGTATTAGT |
| | | 4958-5098 bp | CATCGCTATTACCATGGTGATGCGGTTTTGGCAGT |
| | | | ACATCAATGGGCGTGGATAGCGGTTTGACTCACG |
| | | | GGGATTTCCAAGTCTCCACCCCATTGACGTCAATG |
| | | | GGAGTTTGTTTTGGCACCAAAATCAACGGGACTTT |
| | | | CCAAAATGTCGTAACAACTCCGCCCCATTGACGC |
| | | | AAATGGGCGGTAGGCGTGTACGGTGGGAGGTCTA |
| | | | TATAAGCAGAGCTGGTTTAGTGAACCGTCAGATC |
| | | | CAAGTTTGTACAAAAAAGCAGGCTGCCACCATGA |
| | | | CCGCCTGCGCCCGCCGAGCGGGTGGGCTTCCGGA |
| | | | CCCCGGGCTCTGCGGTCCCGCGTGGTGGGCTCCGT |
| | | | CCCTGCCCCGCCTCCCCCGGGCCCTGCCCCGGCTC |
| | | | CCGCTCCTGCTGCTCCTGCTTCTGCTGCAGCCCCC |
| | | | CGCCCTCTCCGCCGTGTTCACGGTGGGGGTCCTGG |
| | | | GCCCCTGGGCTTGCGACCCCATCTTCTCTCGGGCT |
| | | | CGCCCGGACCTGGCCGCCCGCCTGGCCGCCGCCC |
| | | | GCCTGAACCGCGACCCCGGCCTGGCAGGCGGTCC |
| | | | CCGCTTCGAGGTAGCGCTGCTGCCCGAGCCTTGCC |
| | | | GGACGCCGGGCTCGCTGGGGGCCGTGTCCTCCGC |
| | | | GCTGGCCCGCGTGTCGGGCCTCGTGGGTCCGGTG |
| | | | AACCCTGCGGCCTGCCGGCCAGCCGAGCTGCTCG |
| | | | CCGAAGAAGCCGGGATCGCGCTGGTGCCCTGGGG |
| | | | CTGCCCCTGGACGCAGGCGGAGGGCACCACGGCC |
| | | | CCTGCCGTGACCCCCGCCGCGGATGCCCTCTACGC |
| | | | CCTGCTTCGCGCATTCGGCTGGGCGCGCGTGGCCC |
| | | | TGGTCACCGCCCCCCAGGACCTGTGGGTGGAGGC |
| | | | GGGACGCTCACTGTCCACGGCACTCAGGGCCCGG |
| | | | GGCCTGCCTGTCGCCTCCGTGACTTCCATGGAGCC |
| | | | CTTGGACCTGTCTGGAGCCCGGGAGGCCCTGAGG |
| | | | AAGGTTCGGGACGGGCCCAGGGTCACAGCAGTGA |
| | | | TCATGGTGATGCACTCGGTGCTGCTGGGTGGCGA |
| | | | GGAGCAGCGCTACCTCCTGGAGGCCGCAGAGGAG |
| | | | CTGGGCCTGACCGATGGCTCCCTGGTCTTCCTGCC |
| | | | CTTCGACACGATCCACTACGCCTTGTCCCCAGGCC |
| | | | CGGAGGCCTTGGCCGCACTCGCCAACAGCTCCCA |
| | | | GCTTCGCAGGGCCCACGATGCCGTGCTCACCCTC |
| | | | ACGCGCCACTGTCCCTCTGAAGGCAGCGTGCTGG |
| | | | ACAGCCTGCGCAGGGCTCAAGAGCGCCGCGAGCT |
| | | | GCCCTCTGACCTCAATCTGCAGCAGGTCTCCCCAC |
| | | | TCTTTGGCACCATCTATGACGCGGTCTTCTTGCTG |
| | | | GCAAGGGGCGTGGCAGAAGCGCGGGCTGCCGCA |
| | | | GGTGGCAGATGGGTGTCCGGAGCAGCTGTGGCCC |
| | | | GCCACATCCGGGATGCGCAGGTCCCTGGCTTCTG |
| | | | CGGGGACCTAGGAGGAGACGAGGAGCCCCCATTC |
| | | | GTGCTGCTAGACACGGACGCGGCGGGAGACCGGC |
| | | | TTTTTGCCACATACATGCTGGATCCTGCCCGGGGC |
| | | | TCCTTCCTCTCCGCCGGTACCCGGATGCACTTCCC |
| | | | GCGTGGGGGATCAGCACCCGGACCTGACCCCTCG |
| | | | TGCTGGTTCGATCCAAACAACATCTGCGGTGGAG |
| | | | GACTGGAGCCGGGCCTCGTCTTTCTTGGCTTCCTC |
| | | | CTGGTGGTTGGGATGGGGCTGGCTGGGGCCTTCC |
| | | | TGGCCCATTATGTGAGGCACCGGCTACTTCACATG |
| | | | CAAATGGTCTCCGGCCCCAACAAGATCATCCTGA |
| | | | CCGTGGACGACATCACCTTTCTCCACCCACATGGG |
| | | | GGCACCTCTCGAAAGGTGGCCCAGGGGAGTCGAT |
| | | | CAAGTCTGGGTGCCCGCAGCATGTCAGACATTCG |
| | | | CAGCGGCCCCAGCCAACACTTGGACAGCCCCAAC |
| | | | ATTGGTGTCTATGAGGGAGACAGGGTTTGGCTGA |
| | | | AGAAATTCCCAGGGGATCAGCACATAGCTATCCG |
| | | | CCCAGCAACCAAGACGGCCTTCTCCAAGCTCCAG |
| | | | GAGCTCCGGCATGAGAACGTGGCCCTCTACCTGG |
| | | | GGCTTTTCCTGGCTCGGGGAGCAGAAGGCCCTGC |
| | | | GGCCCTCTGGGAGGGCAACCTGGCTGTGGTCTCA |
| | | | GAGCACTGCACGCGGGGCTCTCTTCAGGACCTCC |
| | | | TCGCTCAGAGAGAAATAAAGCTGGACTGGATGTT |
| | | | CAAGTCCTCCCTCCTGCTGGACCTTATCAAGGGAA |
| | | | TAAGGTATCTGCACCATCGAGGCGTGGCTCATGG |
| | | | GCGGCTGAAGTCACGGAACTGCATAGTGGATGGC |
| | | | AGATTCGTACTCAAGATCACTGACCACGGCCACG |
| | | | GGAGACTGCTGGAAGCACAGAAGGTGCTACCGGA |
| | | | GCCTCCCAGAGCGGAGGACCAGCTGTGGACAGCC |
| | | | CCGGAGCTGCTTAGGGACCCAGCCCTGGAGCGCC |
| | | | GGGGAACGCTGGCCGGCGACGTCTTTAGCTTGGC |
| | | | CATCATCATGCAAGAAGTAGTGTGCCGCAGTGCC |
| | | | CCTTATGCCATGCTGGAGCTCACTCCCGAGGAAG |
| | | | TGGTGCAGAGGGTGCGGAGCCCCCCTCCACTGTG |
| | | | TCGGCCCTTGGTGTCCATGGACCAGGCACCTGTCG |
| | | | AGTGTATCCTCCTGATGAAGCAGTGCTGGGCAGA |
| | | | GCAGCCGGAACTTCGGCCCTCCATGGACCACACC |
| | | | TTCGACCTGTTCAAGAACATCAACAAGGGCCGGA |
| | | | AGACGAACATCATTGACTCGATGCTTCGGATGCT |
| | | | GGAGCAGTACTCTAGTAACCTGGAGGATCTGATC |
| | | | CGGGAGCGCACGGAGGAGCTGGAGCTGGAAAAG |
| | | | CAGAAGACAGACCGGCTGCTTACACAGATGCTGC |
| | | | CTCCGTCTGTGGCTGAGGCCTTGAAGACGGGGAC |
| | | | ACCAGTGGAGCCCGAGTACTTTGAGCAAGTGACA |
| | | | CTGTACTTTAGTGACATTGTGGGCTTCACCACCAT |
| | | | CTCTGCCATGAGTGAGCCCATTGAGGTTGTGGAC |
| | | | CTGCTCAACGATCTCTACACACTCTTTGATGCCAT |
| | | | CATTGGTTCCCACGATGTCTACAAGGTGGAGACA |
| | | | ATAGGGGACGCCTATATGGTGGCCTCGGGGCTGC |
| | | | CCCAGCGGAATGGGCAGCGACACGCGGCAGAGA |
| | | | TCGCCAACATGTCACTGGACATCCTCAGTGCCGTG |
| | | | GGCACTTTCCGCATGCGCCATATGCCTGAGGTTCC |
| | | | CGTGCGCATCCGCATAGGCCTGCACTCGGGTCCA |
| | | | TGCGTGGCAGGCGTGGTGGGCCTCACCATGCCGC |
| | | | GGTACTGCCTGTTTGGGGACACGGTCAACACCGC |
| | | | CTCGCGCATGGAGTCCACCGGGCTGCCTTACCGC |
| | | | ATCCACGTGAACTTGAGCACTGTGGGGATTCTCC |
| | | | GTGCTCTGGACTCGGGCTACCAGGTGGAGCTGCG |
| | | | AGGCCGCACGGAGCTGAAGGGCAAGGGCGCCGA |
| | | | GGACACTTTCTGGCTAGTGGGCAGACGCGGCTTC |
| | | | AACAAGCCCATCCCCAAACCGCCTGACCTGCAAC |
| | | | CGGGGTCCAGCAACCACGGCATCAGCCTGCAGGA |
| | | | GATCCCACCCGAGCGGCGACGGAAGCTGGAGAA |
| | | | GGCGCGGCCGGGCCAGTTCTCTTGAAATCAACCT |
| | | | CTGGATTACAAAATTTGTGAAAGATTGACTGGTA |
| | | | TTCTTAACTATGTTGCTCCTTTTACGCTATGTGGAT |
| | | | ACGCTGCTTTAATGCCTTTGTATCATGCTATTGCT |
| | | | TCCCGTATGGCTTTCATTTTCTCCTCCTTGTATAAA |
| | | | TCCTGGTTGCTGTCTCTTTATGAGGAGTTGTGGCC |
| | | | CGTTGTCAGGCAACGTGGCGTGGTGTGCACTGTG |
| | | | TTTGCTGACGCAACCCCCACTGGTTGGGGCATTGC |
| | | | CACCACCTGTCAGCTCCTTTCCGGGACTTTCGCTT |
| | | | TCCCCCTCCCTATTGCCACGGCGGAACTCATCGCC |
| | | | GCCTGCCTTGCCCGCTGCTGGACAGGGGCTCGGC |
| | | | TGTTGGGCACTGACAATTCCGTGGTGTTGTCGGGG |
| | | | AAATCATCGTCCTTTCCTTGGCTGCTCGCCTGTGT |
| | | | TGCCACCTGGATTCTGCGCGGGACGTCCTTCTGCT |
| | | | ACGTCCCTTCGGCCCTCAATCCAGCGGACCTTCCT |
| | | | TCCCGCGGCCTGCTGCCGGCTCTGCGGCCTCTTCC |
| | | | GCGTCTTCGCCTTCGCCCTCAGACGAGTCGGATCT |
| | | | CCCTTTGGGCCGCCTCCCCGCACCCAGCTTTCTTG |
| | | | TACAAAGTGGGAATTCCTAGAGCTCGCTGATCAG |
| | | | CCTCGACTGTGCCTTCTAGTTGCCAGCCATCTGTT |
| | | | GTTTGCCCCTCCCCCGTGCCTTCCTTGACCCTGGA |
| | | | AGGTGCCACTCCCACTGTCCTTTCCTAATAAAATG |
| | | | AGGAAATTGCATCGCATTGTCTGAGTAGGTGTCA |
| | | | TTCTATTCTGGGGGGTGGGGTGGGGCAGGACAGC |
| | | | AAGGGGGAGGATTGGGAAGAGAATAGCAGGCAT |
| | | | GCTGGGGAGGGCCGCAGGAACCCCTAGTGATGGA |
| | | | GTTGGCCACTCCCTCTCTGCGCGCTCGCTCGCTCA |
| | | | CTGAGGCCGGGCGACCAAAGGTCGCCCGACGCCC |
| | | | GGGCTTTGCCCGGGCGGCCTCAGTGAGCGAGCGA |
| | | | GCGCGCAGCTGCCTGCAGG |
| 4 | AAVss- | AAV2 5′ ITR: | CCTGCAGGCAGCTGCGCGCTCGCTCGCTCACTGA |
| | CMV- | 1-141 bp | GGCCGCCCGGGCAAAGCCCGGGCGTCGGGCGACC |
| | hGUCY2D | CMV: 169-757 | TTTGGTCGCCCGGCCTCAGTGAGCGAGCGAGCGC |
| | | bp | GCAGAGAGGGAGTGGCCAACTCCATCACTAGGGG |
| | | Kozak: 782-787 | TTCCTTCTAGACAACTTTGTATAGAAAAGTTGTAG |
| | | bp | TTATTAATAGTAATCAATTACGGGGTCATTAGTTC |
| | | hGUCY2D [cds | ATAGCCCATATATGGAGTTCCGCGTTACATAACTT |
| | | from | ACGGTAAATGGCCCGCCTGGCTGACCGCCCAACG |
| | | NM_000180.4]: | ACCCCCGCCCATTGACGTCAATAATGACGTATGTT |
| | | 788-4099 bp | CCCATAGTAACGCCAATAGGGACTTTCCATTGAC |
| | | BGH pA: 4154- | GTCAATGGGTGGAGTATTTACGGTAAACTGCCCA |
| | | 4361 bp | CTTGGCAGTACATCAAGTGTATCATATGCCAAGT |
| | | AAV2 3′ ITR: | ACGCCCCCTATTGACGTCAATGACGGTAAATGGC |
| | | 4369-4509 bp | CCGCCTGGCATTATGCCCAGTACATGACCTTATGG |
| | | | GACTTTCCTACTTGGCAGTACATCTACGTATTAGT |
| | | | CATCGCTATTACCATGGTGATGCGGTTTTGGCAGT |
| | | | ACATCAATGGGCGTGGATAGCGGTTTGACTCACG |
| | | | GGGATTTCCAAGTCTCCACCCCATTGACGTCAATG |
| | | | GGAGTTTGTTTTGGCACCAAAATCAACGGGACTTT |
| | | | CCAAAATGTCGTAACAACTCCGCCCCATTGACGC |
| | | | AAATGGGCGGTAGGCGTGTACGGTGGGAGGTCTA |
| | | | TATAAGCAGAGCTGGTTTAGTGAACCGTCAGATC |
| | | | CAAGTTTGTACAAAAAAGCAGGCTGCCACCATGA |
| | | | CCGCCTGCGCCCGCCGAGCGGGTGGGCTTCCGGA |
| | | | CCCCGGGCTCTGCGGTCCCGCGTGGTGGGCTCCGT |
| | | | CCCTGCCCCGCCTCCCCCGGGCCCTGCCCCGGCTC |
| | | | CCGCTCCTGCTGCTCCTGCTTCTGCTGCAGCCCCC |
| | | | CGCCCTCTCCGCCGTGTTCACGGTGGGGGTCCTGG |
| | | | GCCCCTGGGCTTGCGACCCCATCTTCTCTCGGGCT |
| | | | CGCCCGGACCTGGCCGCCCGCCTGGCCGCCGCCC |
| | | | GCCTGAACCGCGACCCCGGCCTGGCAGGCGGTCC |
| | | | CCGCTTCGAGGTAGCGCTGCTGCCCGAGCCTTGCC |
| | | | GGACGCCGGGCTCGCTGGGGGCCGTGTCCTCCGC |
| | | | GCTGGCCCGCGTGTCGGGCCTCGTGGGTCCGGTG |
| | | | AACCCTGCGGCCTGCCGGCCAGCCGAGCTGCTCG |
| | | | CCGAAGAAGCCGGGATCGCGCTGGTGCCCTGGGG |
| | | | CTGCCCCTGGACGCAGGCGGAGGGCACCACGGCC |
| | | | CCTGCCGTGACCCCCGCCGCGGATGCCCTCTACGC |
| | | | CCTGCTTCGCGCATTCGGCTGGGCGCGCGTGGCCC |
| | | | TGGTCACCGCCCCCCAGGACCTGTGGGTGGAGGC |
| | | | GGGACGCTCACTGTCCACGGCACTCAGGGCCCGG |
| | | | GGCCTGCCTGTCGCCTCCGTGACTTCCATGGAGCC |
| | | | CTTGGACCTGTCTGGAGCCCGGGAGGCCCTGAGG |
| | | | AAGGTTCGGGACGGGCCCAGGGTCACAGCAGTGA |
| | | | TCATGGTGATGCACTCGGTGCTGCTGGGTGGCGA |
| | | | GGAGCAGCGCTACCTCCTGGAGGCCGCAGAGGAG |
| | | | CTGGGCCTGACCGATGGCTCCCTGGTCTTCCTGCC |
| | | | CTTCGACACGATCCACTACGCCTTGTCCCCAGGCC |
| | | | CGGAGGCCTTGGCCGCACTCGCCAACAGCTCCCA |
| | | | GCTTCGCAGGGCCCACGATGCCGTGCTCACCCTC |
| | | | ACGCGCCACTGTCCCTCTGAAGGCAGCGTGCTGG |
| | | | ACAGCCTGCGCAGGGCTCAAGAGCGCCGCGAGCT |
| | | | GCCCTCTGACCTCAATCTGCAGCAGGTCTCCCCAC |
| | | | TCTTTGGCACCATCTATGACGCGGTCTTCTTGCTG |
| | | | GCAAGGGGCGTGGCAGAAGCGCGGGCTGCCGCA |
| | | | GGTGGCAGATGGGTGTCCGGAGCAGCTGTGGCCC |
| | | | GCCACATCCGGGATGCGCAGGTCCCTGGCTTCTG |
| | | | CGGGGACCTAGGAGGAGACGAGGAGCCCCCATTC |
| | | | GTGCTGCTAGACACGGACGCGGCGGGAGACCGGC |
| | | | TTTTTGCCACATACATGCTGGATCCTGCCCGGGGC |
| | | | TCCTTCCTCTCCGCCGGTACCCGGATGCACTTCCC |
| | | | GCGTGGGGGATCAGCACCCGGACCTGACCCCTCG |
| | | | TGCTGGTTCGATCCAAACAACATCTGCGGTGGAG |
| | | | GACTGGAGCCGGGCCTCGTCTTTCTTGGCTTCCTC |
| | | | CTGGTGGTTGGGATGGGGCTGGCTGGGGCCTTCC |
| | | | TGGCCCATTATGTGAGGCACCGGCTACTTCACATG |
| | | | CAAATGGTCTCCGGCCCCAACAAGATCATCCTGA |
| | | | CCGTGGACGACATCACCTTTCTCCACCCACATGGG |
| | | | GGCACCTCTCGAAAGGTGGCCCAGGGGAGTCGAT |
| | | | CAAGTCTGGGTGCCCGCAGCATGTCAGACATTCG |
| | | | CAGCGGCCCCAGCCAACACTTGGACAGCCCCAAC |
| | | | ATTGGTGTCTATGAGGGAGACAGGGTTTGGCTGA |
| | | | AGAAATTCCCAGGGGATCAGCACATAGCTATCCG |
| | | | CCCAGCAACCAAGACGGCCTTCTCCAAGCTCCAG |
| | | | GAGCTCCGGCATGAGAACGTGGCCCTCTACCTGG |
| | | | GGCTTTTCCTGGCTCGGGGAGCAGAAGGCCCTGC |
| | | | GGCCCTCTGGGAGGGCAACCTGGCTGTGGTCTCA |
| | | | GAGCACTGCACGCGGGGCTCTCTTCAGGACCTCC |
| | | | TCGCTCAGAGAGAAATAAAGCTGGACTGGATGTT |
| | | | CAAGTCCTCCCTCCTGCTGGACCTTATCAAGGGAA |
| | | | TAAGGTATCTGCACCATCGAGGCGTGGCTCATGG |
| | | | GCGGCTGAAGTCACGGAACTGCATAGTGGATGGC |
| | | | AGATTCGTACTCAAGATCACTGACCACGGCCACG |
| | | | GGAGACTGCTGGAAGCACAGAAGGTGCTACCGGA |
| | | | GCCTCCCAGAGCGGAGGACCAGCTGTGGACAGCC |
| | | | CCGGAGCTGCTTAGGGACCCAGCCCTGGAGCGCC |
| | | | GGGGAACGCTGGCCGGCGACGTCTTTAGCTTGGC |
| | | | CATCATCATGCAAGAAGTAGTGTGCCGCAGTGCC |
| | | | CCTTATGCCATGCTGGAGCTCACTCCCGAGGAAG |
| | | | TGGTGCAGAGGGTGCGGAGCCCCCCTCCACTGTG |
| | | | TCGGCCCTTGGTGTCCATGGACCAGGCACCTGTCG |
| | | | AGTGTATCCTCCTGATGAAGCAGTGCTGGGCAGA |
| | | | GCAGCCGGAACTTCGGCCCTCCATGGACCACACC |
| | | | TTCGACCTGTTCAAGAACATCAACAAGGGCCGGA |
| | | | AGACGAACATCATTGACTCGATGCTTCGGATGCT |
| | | | GGAGCAGTACTCTAGTAACCTGGAGGATCTGATC |
| | | | CGGGAGCGCACGGAGGAGCTGGAGCTGGAAAAG |
| | | | CAGAAGACAGACCGGCTGCTTACACAGATGCTGC |
| | | | CTCCGTCTGTGGCTGAGGCCTTGAAGACGGGGAC |
| | | | ACCAGTGGAGCCCGAGTACTTTGAGCAAGTGACA |
| | | | CTGTACTTTAGTGACATTGTGGGCTTCACCACCAT |
| | | | CTCTGCCATGAGTGAGCCCATTGAGGTTGTGGAC |
| | | | CTGCTCAACGATCTCTACACACTCTTTGATGCCAT |
| | | | CATTGGTTCCCACGATGTCTACAAGGTGGAGACA |
| | | | ATAGGGGACGCCTATATGGTGGCCTCGGGGCTGC |
| | | | CCCAGCGGAATGGGCAGCGACACGCGGCAGAGA |
| | | | TCGCCAACATGTCACTGGACATCCTCAGTGCCGTG |
| | | | GGCACTTTCCGCATGCGCCATATGCCTGAGGTTCC |
| | | | CGTGCGCATCCGCATAGGCCTGCACTCGGGTCCA |
| | | | TGCGTGGCAGGCGTGGTGGGCCTCACCATGCCGC |
| | | | GGTACTGCCTGTTTGGGGACACGGTCAACACCGC |
| | | | CTCGCGCATGGAGTCCACCGGGCTGCCTTACCGC |
| | | | ATCCACGTGAACTTGAGCACTGTGGGGATTCTCC |
| | | | GTGCTCTGGACTCGGGCTACCAGGTGGAGCTGCG |
| | | | AGGCCGCACGGAGCTGAAGGGCAAGGGCGCCGA |
| | | | GGACACTTTCTGGCTAGTGGGCAGACGCGGCTTC |
| | | | AACAAGCCCATCCCCAAACCGCCTGACCTGCAAC |
| | | | CGGGGTCCAGCAACCACGGCATCAGCCTGCAGGA |
| | | | GATCCCACCCGAGCGGCGACGGAAGCTGGAGAA |
| | | | GGCGCGGCCGGGCCAGTTCTCTTGAACCCAGCTTT |
| | | | CTTGTACAAAGTGGGAATTCCTAGAGCTCGCTGA |
| | | | TCAGCCTCGACTGTGCCTTCTAGTTGCCAGCCATC |
| | | | TGTTGTTTGCCCCTCCCCCGTGCCTTCCTTGACCCT |
| | | | GGAAGGTGCCACTCCCACTGTCCTTTCCTAATAAA |
| | | | ATGAGGAAATTGCATCGCATTGTCTGAGTAGGTG |
| | | | TCATTCTATTCTGGGGGGTGGGGTGGGGCAGGAC |
| | | | AGCAAGGGGGAGGATTGGGAAGAGAATAGCAGG |
| | | | CATGCTGGGGAGGGCCGCAGGAACCCCTAGTGAT |
| | | | GGAGTTGGCCACTCCCTCTCTGCGCGCTCGCTCGC |
| | | | TCACTGAGGCCGGGCGACCAAAGGTCGCCCGACG |
| | | | CCCGGGCTTTGCCCGGGCGGCCTCAGTGAGCGAG |
| | | | CGAGCGCGCAGCTGCCTGCAGG |
| 5 | AAV2 5′ | 141 bp | CCTGCAGGCAGCTGCGCGCTCGCTCGCTCACTGA |
| | ITR | | GGCCGCCCGGGCAAAGCCCGGGCGTCGGGCGACC |
| | | | TTTGGTCGCCCGGCCTCAGTGAGCGAGCGAGCGC |
| | | | GCAGAGAGGGAGTGGCCAACTCCATCACTAGGGG |
| | | | TTCCT |
| 6 | AAV2 3′ | 141 bp | AGGAACCCCTAGTGATGGAGTTGGCCACTCCCTC |
| | ITR | | TCTGCGCGCTCGCTCGCTCACTGAGGCCGGGCGA |
| | | | CCAAAGGTCGCCCGACGCCCGGGCTTTGCCCGGG |
| | | | CGGCCTCAGTGAGCGAGCGAGCGCGCAGCTGCCT |
| | | | GCAGG |
| 7 | RK | | TGTAGTTAATGATTAACCCGCCATGCTACTTATCT |
| | promoter | | ACGTACATTTATATTGGCTCATGTCCAACATTACC |
| | | | GCCATGTTGACATTGATTATTGACTAGAATTCGCT |
| | | | AGCAAGATCCAAGCTCAGATCTCGATCGAGTTGG |
| | | | GCCCCAGAAGCCTGGTGGTTGTTTGTCCTTCTCAG |
| | | | GGGAAAAGTGAGGCGGCCCCTTGGAGGAAGGGG |
| | | | CCGGGCAGAATGATCTAATCGGATTCCAAGCAGC |
| | | | TCAGGGGATTGTCTTTTTCTAGCACCTTCTTGCCA |
| | | | CTCCTAAGCGTCCTCCGTGACCCCGGCTGGGATTT |
| | | | AGCCTGGTGCTGTGTCAGCCCCGGTCTCCCAGGG |
| | | | GCTTCCCAGTGGTCCCCAGGAACCCTCGACAGGG |
| | | | CCCGGTCTCTCTCGTCCAGCAAGGGCAGGGACGG |
| | | | GCCACAGGCCAAGGGCCCTCGATCGAGGAACTGA |
| | | | AAAAC |
| 8 | CMV | | TAGTTATTAATAGTAATCAATTACGGGGTCATTAG |
| | promoter | | TTCATAGCCCATATATGGAGTTCCGCGTTACATAA |
| | | | CTTACGGTAAATGGCCCGCCTGGCTGACCGCCCA |
| | | | ACGACCCCCGCCCATTGACGTCAATAATGACGTA |
| | | | TGTTCCCATAGTAACGCCAATAGGGACTTTCCATT |
| | | | GACGTCAATGGGTGGAGTATTTACGGTAAACTGC |
| | | | CCACTTGGCAGTACATCAAGTGTATCATATGCCA |
| | | | AGTACGCCCCCTATTGACGTCAATGACGGTAAAT |
| | | | GGCCCGCCTGGCATTATGCCCAGTACATGACCTTA |
| | | | TGGGACTTTCCTACTTGGCAGTACATCTACGTATT |
| | | | AGTCATCGCTATTACCATGGTGATGCGGTTTTGGC |
| | | | AGTACATCAATGGGCGTGGATAGCGGTTTGACTC |
| | | | ACGGGGATTTCCAAGTCTCCACCCCATTGACGTCA |
| | | | ATGGGAGTTTGTTTTGGCACCAAAATCAACGGGA |
| | | | CTTTCCAAAATGTCGTAACAACTCCGCCCCATTGA |
| | | | CGCAAATGGGCGGTAGGCGTGTACGGTGGGAGGT |
| | | | CTATATAAGCAGAGCTGGTTTAGTGAACCGTCAG |
| | | | ATC |
| 9 | hGUCY2D | | ATGACCGCCTGCGCCCGCCGAGCGGGTGGGCTT |
| | [cds | | CCGGACCCCGGGCTCTGCGGTCCCGCGTGGTGGG |
| | from | | CTCCGTCCCTGCCCCGCCTCCCCCGGGCCCTGCCC |
| | NM_ | | CGGCTCCCGCTCCTGCTGCTCCTGCTTCTGCTGCA |
| | 000180.4] | | GCCCCCCGCCCTCTCCGCCGTGTTCACGGTGGGGG |
| | | | TCCTGGGCCCCTGGGCTTGCGACCCCATCTTCTCT |
| | | | CGGGCTCGCCCGGACCTGGCCGCCCGCCTGGCCG |
| | | | CCGCCCGCCTGAACCGCGACCCCGGCCTGGCAGG |
| | | | CGGTCCCCGCTTCGAGGTAGCGCTGCTGCCCGAG |
| | | | CCTTGCCGGACGCCGGGCTCGCTGGGGGCCGTGT |
| | | | CCTCCGCGCTGGCCCGCGTGTCGGGCCTCGTGGGT |
| | | | CCGGTGAACCCTGCGGCCTGCCGGCCAGCCGAGC |
| | | | TGCTCGCCGAAGAAGCCGGGATCGCGCTGGTGCC |
| | | | CTGGGGCTGCCCCTGGACGCAGGCGGAGGGCACC |
| | | | ACGGCCCCTGCCGTGACCCCCGCCGCGGATGCCC |
| | | | TCTACGCCCTGCTTCGCGCATTCGGCTGGGCGCGC |
| | | | GTGGCCCTGGTCACCGCCCCCCAGGACCTGTGGG |
| | | | TGGAGGCGGGACGCTCACTGTCCACGGCACTCAG |
| | | | GGCCCGGGGCCTGCCTGTCGCCTCCGTGACTTCCA |
| | | | TGGAGCCCTTGGACCTGTCTGGAGCCCGGGAGGC |
| | | | CCTGAGGAAGGTTCGGGACGGGCCCAGGGTCACA |
| | | | GCAGTGATCATGGTGATGCACTCGGTGCTGCTGG |
| | | | GTGGCGAGGAGCAGCGCTACCTCCTGGAGGCCGC |
| | | | AGAGGAGCTGGGCCTGACCGATGGCTCCCTGGTC |
| | | | TTCCTGCCCTTCGACACGATCCACTACGCCTTGTC |
| | | | CCCAGGCCCGGAGGCCTTGGCCGCACTCGCCAAC |
| | | | AGCTCCCAGCTTCGCAGGGCCCACGATGCCGTGC |
| | | | TCACCCTCACGCGCCACTGTCCCTCTGAAGGCAGC |
| | | | GTGCTGGACAGCCTGCGCAGGGCTCAAGAGCGCC |
| | | | GCGAGCTGCCCTCTGACCTCAATCTGCAGCAGGT |
| | | | CTCCCCACTCTTTGGCACCATCTATGACGCGGTCT |
| | | | TCTTGCTGGCAAGGGGCGTGGCAGAAGCGCGGGC |
| | | | TGCCGCAGGTGGCAGATGGGTGTCCGGAGCAGCT |
| | | | GTGGCCCGCCACATCCGGGATGCGCAGGTCCCTG |
| | | | GCTTCTGCGGGGACCTAGGAGGAGACGAGGAGCC |
| | | | CCCATTCGTGCTGCTAGACACGGACGCGGCGGGA |
| | | | GACCGGCTTTTTGCCACATACATGCTGGATCCTGC |
| | | | CCGGGGCTCCTTCCTCTCCGCCGGTACCCGGATGC |
| | | | ACTTCCCGCGTGGGGGATCAGCACCCGGACCTGA |
| | | | CCCCTCGTGCTGGTTCGATCCAAACAACATCTGCG |
| | | | GTGGAGGACTGGAGCCGGGCCTCGTCTTTCTTGG |
| | | | CTTCCTCCTGGTGGTTGGGATGGGGCTGGCTGGG |
| | | | GCCTTCCTGGCCCATTATGTGAGGCACCGGCTACT |
| | | | TCACATGCAAATGGTCTCCGGCCCCAACAAGATC |
| | | | ATCCTGACCGTGGACGACATCACCTTTCTCCACCC |
| | | | ACATGGGGGCACCTCTCGAAAGGTGGCCCAGGGG |
| | | | AGTCGATCAAGTCTGGGTGCCCGCAGCATGTCAG |
| | | | ACATTCGCAGCGGCCCCAGCCAACACTTGGACAG |
| | | | CCCCAACATTGGTGTCTATGAGGGAGACAGGGTT |
| | | | TGGCTGAAGAAATTCCCAGGGGATCAGCACATAG |
| | | | CTATCCGCCCAGCAACCAAGACGGCCTTCTCCAA |
| | | | GCTCCAGGAGCTCCGGCATGAGAACGTGGCCCTC |
| | | | TACCTGGGGCTTTTCCTGGCTCGGGGAGCAGAAG |
| | | | GCCCTGCGGCCCTCTGGGAGGGCAACCTGGCTGT |
| | | | GGTCTCAGAGCACTGCACGCGGGGCTCTCTTCAG |
| | | | GACCTCCTCGCTCAGAGAGAAATAAAGCTGGACT |
| | | | GGATGTTCAAGTCCTCCCTCCTGCTGGACCTTATC |
| | | | AAGGGAATAAGGTATCTGCACCATCGAGGCGTGG |
| | | | CTCATGGGCGGCTGAAGTCACGGAACTGCATAGT |
| | | | GGATGGCAGATTCGTACTCAAGATCACTGACCAC |
| | | | GGCCACGGGAGACTGCTGGAAGCACAGAAGGTG |
| | | | CTACCGGAGCCTCCCAGAGCGGAGGACCAGCTGT |
| | | | GGACAGCCCCGGAGCTGCTTAGGGACCCAGCCCT |
| | | | GGAGCGCCGGGGAACGCTGGCCGGCGACGTCTTT |
| | | | AGCTTGGCCATCATCATGCAAGAAGTAGTGTGCC |
| | | | GCAGTGCCCCTTATGCCATGCTGGAGCTCACTCCC |
| | | | GAGGAAGTGGTGCAGAGGGTGCGGAGCCCCCCTC |
| | | | CACTGTGTCGGCCCTTGGTGTCCATGGACCAGGC |
| | | | ACCTGTCGAGTGTATCCTCCTGATGAAGCAGTGCT |
| | | | GGGCAGAGCAGCCGGAACTTCGGCCCTCCATGGA |
| | | | CCACACCTTCGACCTGTTCAAGAACATCAACAAG |
| | | | GGCCGGAAGACGAACATCATTGACTCGATGCTTC |
| | | | GGATGCTGGAGCAGTACTCTAGTAACCTGGAGGA |
| | | | TCTGATCCGGGAGCGCACGGAGGAGCTGGAGCTG |
| | | | GAAAAGCAGAAGACAGACCGGCTGCTTACACAG |
| | | | ATGCTGCCTCCGTCTGTGGCTGAGGCCTTGAAGAC |
| | | | GGGGACACCAGTGGAGCCCGAGTACTTTGAGCAA |
| | | | GTGACACTGTACTTTAGTGACATTGTGGGCTTCAC |
| | | | CACCATCTCTGCCATGAGTGAGCCCATTGAGGTTG |
| | | | TGGACCTGCTCAACGATCTCTACACACTCTTTGAT |
| | | | GCCATCATTGGTTCCCACGATGTCTACAAGGTGG |
| | | | AGACAATAGGGGACGCCTATATGGTGGCCTCGGG |
| | | | GCTGCCCCAGCGGAATGGGCAGCGACACGCGGCA |
| | | | GAGATCGCCAACATGTCACTGGACATCCTCAGTG |
| | | | CCGTGGGCACTTTCCGCATGCGCCATATGCCTGAG |
| | | | GTTCCCGTGCGCATCCGCATAGGCCTGCACTCGG |
| | | | GTCCATGCGTGGCAGGCGTGGTGGGCCTCACCAT |
| | | | GCCGCGGTACTGCCTGTTTGGGGACACGGTCAAC |
| | | | ACCGCCTCGCGCATGGAGTCCACCGGGCTGCCTT |
| | | | ACCGCATCCACGTGAACTTGAGCACTGTGGGGAT |
| | | | TCTCCGTGCTCTGGACTCGGGCTACCAGGTGGAG |
| | | | CTGCGAGGCCGCACGGAGCTGAAGGGCAAGGGC |
| | | | GCCGAGGACACTTTCTGGCTAGTGGGCAGACGCG |
| | | | GCTTCAACAAGCCCATCCCCAAACCGCCTGACCT |
| | | | GCAACCGGGGTCCAGCAACCACGGCATCAGCCTG |
| | | | CAGGAGATCCCACCCGAGCGGCGACGGAAGCTGG |
| | | | AGAAGGCGCGGCCGGGCCAGTTCTCTTGA |
| 10 | WPRE | | AATCAACCTCTGGATTACAAAATTTGTGAAAGAT |
| | (mut6) | | TGACTGGTATTCTTAACTATGTTGCTCCTTTTACG |
| | | | CTATGTGGATACGCTGCTTTAATGCCTTTGTATCA |
| | | | TGCTATTGCTTCCCGTATGGCTTTCATTTTCTCCTC |
| | | | CTTGTATAAATCCTGGTTGCTGTCTCTTTATGAGG |
| | | | AGTIGTGGCCCGTTGTCAGGCAACGTGGCGTGGT |
| | | | GTGCACTGTGTTTGCTGACGCAACCCCCACTGGTT |
| | | | GGGGCATTGCCACCACCTGTCAGCTCCTTTCCGGG |
| | | | ACTTTCGCTTTCCCCCTCCCTATTGCCACGGCGGA |
| | | | ACTCATCGCCGCCTGCCTTGCCCGCTGCTGGACAG |
| | | | GGGCTCGGCTGTTGGGCACTGACAATTCCGTGGT |
| | | | GTTGTCGGGGAAATCATCGTCCTTTCCTTGGCTGC |
| | | | TCGCCTGTGTTGCCACCTGGATTCTGCGCGGGACG |
| | | | TCCTTCTGCTACGTCCCTTCGGCCCTCAATCCAGC |
| | | | GGACCTTCCTTCCCGCGGCCTGCTGCCGGCTCTGC |
| | | | GGCCTCTTCCGCGTCTTCGCCTTCGCCCTCAGACG |
| | | | AGTCGGATCTCCCTTTGGGCCGCCTCCCCGC |
| 11 | BGH pA | | CTGTGCCTTCTAGTTGCCAGCCATCTGTTGTTTGC |
| | | | CCCTCCCCCGTGCCTTCCTTGACCCTGGAAGGTGC |
| | | | CACTCCCACTGTCCTTTCCTAATAAAATGAGGAAA |
| | | | TTGCATCGCATTGTCTGAGTAGGTGTCATTCTATT |
| | | | CTGGGGGGTGGGGTGGGGCAGGACAGCAAGGGG |
| | | | GAGGATTGGGAAGAGAATAGCAGGCATGCTGGG |
| | | | GA |
| 12 | hRetGC1 | | MTACARRAGGLPDPGLCGPAWWAPSLPRLPRALPR |
| | [NM_ | | LPLLLLLLLLQPPALSAVFTVGVLGPWACDPIFSRAR |
| | 000180.4] | | PDLAARLAAARLNRDPGLAGGPRFEVALLPEPCRTP |
| | | | GSLGAVSSALARVSGLVGPVNPAACRPAELLAEEA |
| | | | GIALVPWGCPWTQAEGTTAPAVTPAADALYALLRA |
| | | | FGWARVALVTAPQDLWVEAGRSLSTALRARGLPV |
| | | | ASVTSMEPLDLSGAREALRKVRDGPRVTAVIMVMH |
| | | | SVLLGGEEQRYLLEAAEELGLTDGSLVFLPFDTIHY |
| | | | ALSPGPEALAALANSSQLRRAHDAVLTLTRHCPSEG |
| | | | SVLDSLRRAQERRELPSDLNLQQVSPLFGTIYDAVFL |
| | | | LARGVAEARAAAGGRWVSGAAVARHIRDAQVPGF |
| | | | CGDLGGDEEPPFVLLDTDAAGDRLFATYMLDPARG |
| | | | SFLSAGTRMHFPRGGSAPGPDPSCWFDPNNICGGGL |
| | | | EPGLVFLGFLLVVGMGLAGAFLAHYVRHRLLHMQ |
| | | | MVSGPNKIILTVDDITFLHPHGGTSRKVAQGSRSSLG |
| | | | ARSMSDIRSGPSQHLDSPNIGVYEGDRVWLKKFPGD |
| | | | QHIAIRPATKTAFSKLQELRHENVALYLGLFLARGA |
| | | | EGPAALWEGNLAVVSEHCTRGSLQDLLAQREIKLD |
| | | | WMFKSSLLLDLIKGIRYLHHRGVAHGRLKSRNCIVD |
| | | | GRFVLKITDHGHGRLLEAQKVLPEPPRAEDQLWTAP |
| | | | ELLRDPALERRGTLAGDVFSLAIIMQEVVCRSAPYA |
| | | | MLELTPEEVVQRVRSPPPLCRPLVSMDQAPVECILL |
| | | | MKQCWAEQPELRPSMDHTFDLFKNINKGRKTNIIDS |
| | | | MLRMLEQYSSNLEDLIRERTEELELEKQKTDRLLTQ |
| | | | MLPPSVAEALKTGTPVEPEYFEQVTLYFSDIVGFTTI |
| | | | SAMSEPIEVVDLLNDLYTLFDAIIGSHDVYKVETIGD |
| | | | AYMVASGLPQRNGQRHAAEIANMSLDILSAVGTFR |
| | | | MRHMPEVPVRIRIGLHSGPCVAGVVGLTMPRYCLF |
| | | | GDTVNTASRMESTGLPYRIHVNLSTVGILRALDSGY |
| | | | QVELRGRTELKGKGAEDTFWLVGRRGFNKPIPKPPD |
| | | | LQPGSSNHGISLQEIPPERRRKLEKARPGQFS |
| 13 | hGUCY2D | | ATGACAGCCTGTGCCAGGAGAGCTGGTGGGCTTC |
| | (Example | | CTGACCCTGGGCTCTGTGGTCCAGCTTGGTGGGCT |
| | 1 for | | CCCTCCCTGCCCAGACTCCCCAGGGCCCTGCCCAG |
| | codon | | GCTCCCTCTCCTGCTGCTCCTGCTTCTGCTGCAGC |
| | optimized | | CCCCTGCCCTCAGTGCAGTGTTCACTGTGGGGGTC |
| | sequence) | | CTGGGCCCCTGGGCTTGTGACCCCATCTTCTCTAG |
| | | | GGCTAGACCTGACCTGGCTGCCAGGCTGGCTGCA |
| | | | GCCAGGCTGAACAGGGACCCTGGCCTGGCAGGGG |
| | | | GTCCCAGGTTTGAGGTAGCCCTGCTGCCAGAGCC |
| | | | TTGCAGGACACCAGGCTCCCTGGGGGCAGTGTCC |
| | | | TCTGCCCTGGCCAGAGTGTCAGGCCTAGTGGGTC |
| | | | CTGTGAACCCTGCAGCCTGCAGACCAGCAGAGCT |
| | | | GCTGGCTGAAGAAGCTGGGATAGCACTGGTGCCC |
| | | | TGGGGCTGCCCCTGGACTCAGGCTGAGGGCACCA |
| | | | CAGCCCCTGCAGTGACCCCAGCTGCAGATGCCCT |
| | | | CTATGCCCTGCTTAGAGCATTTGGCTGGGCCAGA |
| | | | GTGGCCCTGGTCACTGCCCCTCAGGACCTGTGGGT |
| | | | GGAGGCAGGAAGGTCACTGTCCACAGCACTCAGG |
| | | | GCCAGAGGCCTGCCTGTGGCCTCTGTGACTTCCAT |
| | | | GGAGCCCTTGGACCTGTCTGGAGCCAGAGAGGCC |
| | | | CTGAGGAAGGTTAGAGATGGGCCCAGGGTCACAG |
| | | | CAGTGATCATGGTGATGCACAGTGTGCTGCTGGG |
| | | | TGGAGAGGAGCAGAGGTACCTCCTGGAGGCTGCA |
| | | | GAGGAGCTGGGCCTGACAGATGGCTCCCTGGTCT |
| | | | TCCTGCCCTTTGACACCATCCACTATGCCTTGTCC |
| | | | CCAGGCCCAGAGGCCTTGGCTGCACTAGCCAACA |
| | | | GCTCCCAGCTTAGAAGGGCCCATGATGCAGTGCT |
| | | | CACCCTCACCAGACACTGTCCCTCTGAAGGCTCA |
| | | | GTGCTGGACAGCCTGAGAAGGGCTCAAGAGAGG |
| | | | AGAGAGCTGCCCTCTGACCTCAATCTGCAGCAGG |
| | | | TCTCCCCACTCTTTGGCACCATCTATGATGCTGTC |
| | | | TTCTTGCTGGCAAGGGGAGTGGCAGAAGCCAGAG |
| | | | CTGCTGCAGGTGGCAGATGGGTGTCAGGAGCAGC |
| | | | TGTGGCCAGGCACATCAGGGATGCCCAGGTCCCT |
| | | | GGCTTCTGTGGGGACCTAGGAGGAGATGAGGAGC |
| | | | CCCCATTTGTGCTGCTAGACACAGATGCTGCAGG |
| | | | AGACAGGCTTTTTGCCACATACATGCTGGATCCTG |
| | | | CCAGGGGCTCCTTCCTCAGTGCAGGTACCAGGAT |
| | | | GCACTTCCCAAGAGGGGGATCAGCACCTGGACCT |
| | | | GACCCCAGCTGCTGGTTTGATCCAAACAACATCT |
| | | | GTGGTGGAGGACTGGAGCCTGGCCTTGTCTTTCTT |
| | | | GGCTTCCTCCTGGTGGTTGGGATGGGGCTGGCTG |
| | | | GGGCCTTCCTGGCCCATTATGTGAGGCACAGGCT |
| | | | ACTTCACATGCAAATGGTCTCAGGCCCCAACAAG |
| | | | ATCATCCTGACTGTGGATGACATCACCTTTCTCCA |
| | | | CCCACATGGGGGCACCTCTAGAAAGGTGGCCCAG |
| | | | GGGAGTAGATCAAGTCTGGGTGCCAGGAGCATGT |
| | | | CAGACATTAGGTCTGGCCCCAGCCAACACTTGGA |
| | | | CAGCCCCAACATTGGTGTCTATGAGGGAGACAGG |
| | | | GTTTGGCTGAAGAAATTCCCAGGGGATCAGCACA |
| | | | TAGCTATCAGGCCAGCAACCAAGACAGCCTTCTC |
| | | | CAAGCTCCAGGAGCTCAGGCATGAGAATGTGGCC |
| | | | CTCTACCTGGGGCTTTTCCTGGCTAGGGGAGCAG |
| | | | AAGGCCCTGCAGCCCTCTGGGAGGGCAACCTGGC |
| | | | TGTGGTCTCAGAGCACTGCACCAGGGGCTCTCTTC |
| | | | AGGACCTCCTTGCTCAGAGAGAAATAAAGCTGGA |
| | | | CTGGATGTTCAAGTCCTCCCTCCTGCTGGACCTTA |
| | | | TCAAGGGAATAAGGTATCTGCACCATAGGGGAGT |
| | | | GGCTCATGGGAGACTGAAGTCAAGAAACTGCATA |
| | | | GTGGATGGCAGATTTGTACTCAAGATCACTGACC |
| | | | ATGGCCATGGGAGACTGCTGGAAGCACAGAAGGT |
| | | | GCTACCTGAGCCTCCCAGAGCTGAGGACCAGCTG |
| | | | TGGACAGCCCCTGAGCTGCTTAGGGACCCAGCCC |
| | | | TGGAGAGGAGAGGAACCCTGGCAGGAGATGTCTT |
| | | | TAGCTTGGCCATCATCATGCAAGAAGTAGTGTGC |
| | | | AGAAGTGCCCCTTATGCCATGCTGGAGCTCACTCC |
| | | | TGAGGAAGTGGTGCAGAGGGTGAGAAGTCCCCCT |
| | | | CCACTGTGTAGGCCCTTGGTGTCCATGGACCAGG |
| | | | CACCTGTTGAGTGTATCCTCCTGATGAAGCAGTGC |
| | | | TGGGCAGAGCAGCCTGAACTTAGACCCTCCATGG |
| | | | ACCACACCTTTGACCTGTTCAAGAACATCAACAA |
| | | | GGGCAGGAAGACCAACATCATTGACTCAATGCTT |
| | | | AGAATGCTGGAGCAGTACTCTAGTAACCTGGAGG |
| | | | ATCTGATCAGGGAGAGGACAGAGGAGCTGGAGCT |
| | | | GGAAAAGCAGAAGACAGACAGACTGCTTACACA |
| | | | GATGCTGCCTCCTTCTGTGGCTGAGGCCTTGAAGA |
| | | | CAGGGACACCAGTGGAGCCTGAGTACTTTGAGCA |
| | | | AGTGACACTGTACTTTAGTGACATTGTGGGCTTCA |
| | | | CCACCATCTCTGCCATGAGTGAGCCCATTGAGGTT |
| | | | GTGGACCTGCTCAATGATCTCTACACACTCTTTGA |
| | | | TGCCATCATTGGTTCCCATGATGTCTACAAGGTGG |
| | | | AGACAATAGGGGATGCCTATATGGTGGCCTCTGG |
| | | | GCTGCCCCAGAGGAATGGGCAGAGGCATGCTGCA |
| | | | GAGATTGCCAACATGTCACTGGACATCCTCAGTG |
| | | | CTGTGGGCACTTTCAGGATGAGGCATATGCCTGA |
| | | | GGTTCCAGTGAGGATCAGAATAGGCCTGCACAGT |
| | | | GGTCCATGTGTGGCAGGGGTGGTGGGCCTCACCA |
| | | | TGCCCAGGTACTGCCTGTTTGGGGACACAGTCAA |
| | | | CACTGCCAGTAGAATGGAGTCCACTGGGCTGCCT |
| | | | TACAGAATCCATGTGAACTTGAGCACTGTGGGGA |
| | | | TTCTCAGGGCTCTGGACAGTGGCTACCAGGTGGA |
| | | | GCTGAGGGGCAGGACTGAGCTGAAGGGCAAGGG |
| | | | GGCAGAGGACACTTTCTGGCTAGTGGGCAGAAGA |
| | | | GGCTTCAACAAGCCCATCCCCAAACCCCCTGACC |
| | | | TGCAACCAGGGTCCAGCAACCATGGCATCAGCCT |
| | | | GCAGGAGATCCCACCTGAGAGAAGGAGAAAGCT |
| | | | GGAGAAGGCCAGGCCAGGCCAGTTCTCTTGA |
| 14 | hGUCY2D | | ATGACAGCCTGTGCCAGAAGGGCAGGTGGGCTTC |
| | (Example | | CAGACCCAGGGCTCTGTGGTCCTGCTTGGTGGGCT |
| | 2 for | | CCCTCCCTGCCCAGACTCCCCAGAGCCCTGCCCAG |
| | codon | | GCTCCCCCTCCTGCTGCTCCTGCTTCTGCTGCAGC |
| | optimized | | CCCCAGCCCTCAGTGCTGTGTTCACAGTGGGGGTC |
| | sequence) | | CTGGGCCCCTGGGCTTGTGACCCCATCTTCTCTAG |
| | | | GGCTAGGCCTGACCTGGCAGCCAGGCTGGCAGCT |
| | | | GCCAGACTGAACAGGGACCCTGGCCTGGCAGGAG |
| | | | GTCCCAGGTTTGAGGTAGCTCTGCTGCCAGAGCCT |
| | | | TGCAGAACACCTGGCAGTCTGGGGGCTGTGTCCA |
| | | | GTGCACTGGCCAGAGTGTCAGGCTTGGTGGGTCC |
| | | | TGTGAACCCTGCAGCCTGCAGACCAGCTGAGCTG |
| | | | CTGGCTGAAGAAGCTGGGATTGCTCTGGTGCCCT |
| | | | GGGGCTGCCCCTGGACCCAGGCTGAGGGCACCAC |
| | | | AGCCCCTGCTGTGACCCCAGCTGCAGATGCCCTCT |
| | | | ATGCCCTGCTTAGGGCATTTGGCTGGGCCAGGGT |
| | | | GGCCCTGGTCACAGCACCCCAGGACCTGTGGGTG |
| | | | GAGGCTGGAAGGTCACTGTCCACTGCACTCAGGG |
| | | | CCAGGGGCCTGCCTGTGGCCTCAGTGACTTCCATG |
| | | | GAGCCCTTGGACCTGTCTGGAGCCAGGGAGGCCC |
| | | | TGAGGAAGGTTAGAGATGGGCCCAGGGTCACAGC |
| | | | AGTGATCATGGTGATGCACAGTGTGCTGCTGGGT |
| | | | GGTGAGGAGCAGAGGTACCTCCTGGAGGCTGCAG |
| | | | AGGAGCTGGGCCTGACAGATGGCTCCCTGGTCTT |
| | | | CCTGCCCTTTGACACCATCCACTATGCCTTGTCCC |
| | | | CAGGCCCTGAGGCCTTGGCTGCACTGGCCAACAG |
| | | | CTCCCAGCTTAGAAGGGCCCATGATGCTGTGCTC |
| | | | ACCCTCACTAGACACTGTCCCTCTGAAGGCAGTGT |
| | | | GCTGGACAGCCTGAGAAGGGCTCAAGAGAGAAG |
| | | | GGAGCTGCCCTCTGACCTCAATCTGCAGCAGGTCT |
| | | | CCCCACTCTTTGGCACCATCTATGATGCTGTCTTC |
| | | | TTGCTGGCAAGGGGTGTGGCAGAAGCCAGAGCTG |
| | | | CTGCAGGTGGCAGATGGGTGTCTGGAGCAGCTGT |
| | | | GGCCAGGCACATCAGGGATGCACAGGTCCCTGGC |
| | | | TTCTGTGGGGACTTGGGAGGAGATGAGGAGCCCC |
| | | | CATTTGTGCTGCTGGACACAGATGCTGCAGGAGA |
| | | | CAGACTTTTTGCCACATACATGCTGGATCCTGCCA |
| | | | GGGGCTCCTTCCTCTCTGCTGGTACCAGAATGCAC |
| | | | TTCCCTAGAGGGGGATCAGCACCTGGACCTGACC |
| | | | CCTCATGCTGGTTTGATCCAAACAACATCTGTGGT |
| | | | GGAGGACTGGAGCCAGGCCTTGTCTTTCTTGGCTT |
| | | | CCTCCTGGTGGTTGGGATGGGGCTGGCTGGGGCC |
| | | | TTCCTGGCCCATTATGTGAGGCACAGGTTGCTTCA |
| | | | CATGCAAATGGTCTCAGGCCCCAACAAGATCATC |
| | | | CTGACTGTGGATGACATCACCTTTCTCCACCCACA |
| | | | TGGGGGCACCTCTAGAAAGGTGGCCCAGGGGAGT |
| | | | AGATCAAGTCTGGGTGCCAGAAGCATGTCAGACA |
| | | | TTAGGAGTGGCCCCAGCCAACACTTGGACAGCCC |
| | | | CAACATTGGTGTCTATGAGGGAGACAGGGTTTGG |
| | | | CTGAAGAAATTCCCAGGGGATCAGCACATAGCTA |
| | | | TCAGACCAGCAACCAAGACTGCCTTCTCCAAGCT |
| | | | CCAGGAGCTCAGACATGAGAATGTGGCCCTCTAC |
| | | | CTGGGGCTTTTCCTGGCTAGGGGAGCAGAAGGCC |
| | | | CTGCTGCCCTCTGGGAGGGCAACCTGGCTGTGGT |
| | | | CTCAGAGCACTGCACTAGAGGCTCTCTTCAGGAC |
| | | | CTCCTTGCTCAGAGAGAAATAAAGCTGGACTGGA |
| | | | TGTTCAAGTCCTCCCTCCTGCTGGACCTTATCAAG |
| | | | GGAATAAGGTATCTGCACCATAGGGGTGTGGCTC |
| | | | ATGGGAGGCTGAAGTCAAGAAACTGCATAGTGGA |
| | | | TGGCAGATTTGTACTCAAGATCACTGACCATGGC |
| | | | CATGGGAGACTGCTGGAAGCACAGAAGGTGCTGC |
| | | | CAGAGCCTCCCAGAGCAGAGGACCAGCTGTGGAC |
| | | | AGCCCCTGAGCTGCTTAGGGACCCAGCCCTGGAG |
| | | | AGAAGGGGAACACTGGCTGGAGATGTCTTTAGCT |
| | | | TGGCCATCATCATGCAAGAAGTAGTGTGCAGGAG |
| | | | TGCCCCTTATGCCATGCTGGAGCTCACTCCAGAGG |
| | | | AAGTGGTGCAGAGGGTGAGAAGCCCACCTCCACT |
| | | | GTGTAGACCCTTGGTGTCCATGGACCAGGCACCT |
| | | | GTGGAGTGTATCCTCCTGATGAAGCAGTGCTGGG |
| | | | CAGAGCAGCCTGAACTTAGGCCCTCCATGGACCA |
| | | | CACCTTTGACCTGTTCAAGAACATCAACAAGGGC |
| | | | AGAAAGACCAACATCATTGACTCAATGCTTAGAA |
| | | | TGCTGGAGCAGTACTCTAGTAACCTGGAGGATCT |
| | | | GATCAGGGAGAGGACTGAGGAGCTGGAGCTGGA |
| | | | AAAGCAGAAGACAGACAGACTGCTTACACAGATG |
| | | | CTGCCTCCCTCTGTGGCTGAGGCCTTGAAGACAG |
| | | | GGACACCAGTGGAGCCTGAGTACTTTGAGCAAGT |
| | | | GACACTGTACTTTAGTGACATTGTGGGCTTCACCA |
| | | | CCATCTCTGCCATGAGTGAGCCCATTGAGGTTGTG |
| | | | GACCTGCTCAATGATCTCTACACACTCTTTGATGC |
| | | | CATCATTGGTTCCCATGATGTCTACAAGGTGGAG |
| | | | ACAATAGGGGATGCCTATATGGTGGCCTCTGGGC |
| | | | TGCCCCAGAGGAATGGGCAGAGGCATGCTGCAGA |
| | | | GATTGCCAACATGTCACTGGACATCCTCAGTGCTG |
| | | | TGGGCACTTTCAGGATGAGACATATGCCTGAGGT |
| | | | TCCTGTGAGGATCAGGATAGGCCTGCACTCTGGT |
| | | | CCATGTGTGGCAGGAGTGGTGGGCCTCACCATGC |
| | | | CTAGATACTGCCTGTTTGGGGACACAGTCAACAC |
| | | | AGCCTCCAGGATGGAGTCCACAGGGCTGCCTTAC |
| | | | AGGATCCATGTGAACTTGAGCACTGTGGGGATTC |
| | | | TCAGGGCTCTGGACTCAGGCTACCAGGTGGAGCT |
| | | | GAGGGGCAGGACTGAGCTGAAGGGCAAGGGAGC |
| | | | TGAGGACACTTTCTGGCTTGTGGGCAGAAGGGGC |
| | | | TTCAACAAGCCCATCCCCAAACCACCTGACCTGC |
| | | | AACCAGGGTCCAGCAACCATGGCATCAGCCTGCA |
| | | | GGAGATCCCACCTGAGAGGAGAAGGAAGCTGGA |
| | | | GAAGGCAAGGCCAGGCCAGTTCTCTTGA |

While the foregoing written description of the invention enables one of ordinary skill to make and use what is considered presently to be the best mode thereof, those of ordinary skill will understand and appreciate the existence of variations, combinations, and equivalents of the specific embodiments, methods, and examples herein.

We claim:

1. An expression construct comprising:
(a) a promotor sequence that confers expression in photoreceptor cells, and
(b) a nucleic acid sequence encoding retinal membrane guanylyl cyclase 1 (RetGC1);
wherein the nucleic acid sequence is operably linked to the promoter.

2. The expression construct of claim 1, wherein the promotor sequence is a rhodopsin kinase (RK) or a cytomegalovirus (CMV) promotor sequence.

3. The expression construct of claim 2, wherein the promoter sequence comprises a sequence that is at least 90% identical to SEQ ID NO:7.

4. The expression construct of claim 3, wherein the promoter sequence SEQ ID NO:7.

5. The expression construct of claim 2, wherein the promoter sequence comprises a sequence that is at least 90% identical to SEQ ID NO:8.

6. The expression construct of claim 5, wherein the promoter sequence comprises SEQ ID NO:8.

7. The expression construct of any one of the preceding claims, wherein the expression construct further comprises a post transcriptional regulatory element.

8. The expression construct of claim 7, wherein the post transcriptional regulatory element comprises a woodchuck hepatitis virus post transcriptional regulatory element (WPRE).

9. The expression construct of claim 7, wherein the post transcriptional regulatory element comprises a sequence that is at least 90% identical to SEQ ID NO:10.

10. The expression construct of claim 9, wherein the post transcriptional regulatory element comprises SEQ ID NO:10.

11. The expression construct of any one of the claims 1-10, wherein the nucleic acid sequence encoding the RetGC1 is a wildtype RetGC1 gene.

12. The expression construct of any one of the claims 1-10, wherein the nucleic acid sequence encoding the RetGC1 is a codon-optimized sequence.

13. The expression construct of any one of the claims 1-10, wherein the nucleic acid sequence encoding the RetGC1 comprises a sequence that is at least 90% identical to SEQ ID NO:9, SEQ ID NO:13, or SEQ ID NO:14.

14. The expression construct of claim 13, wherein the nucleic acid sequence encoding the RetGC1 comprises SEQ ID NO:9, SEQ ID NO:13, or SEQ ID NO:14.

15. The expression construct of any one of the claims 1-10, wherein the nucleic acid sequence encoding the RetGC1 encodes a protein comprising a sequence that is at least 90% identical to SEQ ID NO:12.

16. The expression construct of claim 15, wherein the nucleic acid sequence encoding the RetGC1 encodes a protein comprising SEQ ID NO:12.

17. The expression construct of any one of the preceding claims, wherein the expression construct further comprises a polyadenylation signal.

18. The expression construct of claim 17, wherein the polyadenylation signal comprises a bovine growth hormone polyadenylation (BGH-polyA) signal.

19. The expression construct of claim 17, wherein the polyadenylation signal comprises a sequence that is at least 90% identical to SEQ ID NO:11.

20. The expression construct of claim 19, wherein the polyadenylation signal comprises SEQ ID NO:11.

21. The expression construct of any one of the preceding claims, wherein the expression construct comprises a sequence that is at least 90% identical to a sequence selected from the group consisting of SEQ ID NOS:1-4.

22. The expression construct of any claim 21, wherein the expression construct comprises a sequence selected from the group consisting of SEQ ID NOS:1-4.

23. A vector comprising the expression construct of any one of the preceding claims.

24. The vector of claim 23, wherein the vector is a viral vector.

25. The vector of claim 24, wherein the vector is an adeno-associated virus (AAV) vector.

26. The vector of claim 25, wherein the vector comprises a genome derived from AAV serotype AAV2.

27. The vector of any one of claims 25 or 26, wherein the vector comprises a capsid derived from AAV7m8.

28. A pharmaceutical composition comprising the vector of any one of claims 23-27 and a pharmaceutically acceptable carrier.

29. A method for treating a retinal disease in a subject in need thereof, wherein the retinal disease is associated with one or more mutations in the GUCY2D gene, the method comprising administering to the subject the vector of any one of claims 23-27 or the pharmaceutical composition of claim 28.

30. The method of claim 29, wherein the retinal disease is cone-rod dystrophy (CRD) or Leber congenital amaurosis type 1 (LCA1).

31. The method of claim 30, wherein the retinal disease is LCA1.

32. A method of increasing expression of rod cGMP-specific 3′,5′-cyclic phosphodiesterase subunit β (PDE6β) in a subject in need thereof, the method comprising administering to the subject the vector of any one of claims 23-27 or the pharmaceutical composition of claim 28.

33. A method of increasing cyclic guanosine monophosphate (cGMP) levels in a photoreceptor in a subject in need thereof, the method comprising administering to the subject the vector of any one of claims 23-27 or the pharmaceutical composition of claim 28.

34. The method of any of claims 29-33, wherein the vector or the pharmaceutical composition is administered by intraocular injection.

35. The method of claim 34, wherein the vector or the pharmaceutical composition is injected into the central retina of the subject.


SEQUENCE LISTING (14 sequences)

SEQ ID NO: 1 - DNA, 5129 bp, synthetic construct
  source 1..5129: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtgtagttaatgattaacccgccatgctacttatctacgtacatttatattggctcatgtccaacattaccgccatgttgacattgattattgactagaattcgctagcaagatccaagctcagatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaagtttgtacaaaaaagcaggctgccaccatgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 4540 bp, synthetic construct
  source 1..4540: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtgtagttaatgattaacccgccatgctacttatctacgtacatttatattggctcatgtccaacattaccgccatgttgacattgattattgactagaattcgctagcaagatccaagctcagatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaagtttgtacaaaaaagcaggctgccaccatgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttgaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 5098 bp, synthetic construct
  source 1..5098: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 4509 bp, synthetic construct
  source 1..4509: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttgaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 452 bp, synthetic construct
  source 1..452: mol_type = other DNA
tgtagttaatgattaacccgccatgctacttatctacgtacatttatattggctcatgtccaacattaccgccatgttgacattgattattgactagaattcgctagcaagatccaagctcagatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgatcgaggaactgaaaaac

SEQ ID NO: 8 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 9 - DNA, 3312 bp, synthetic construct
  source 1..3312: mol_type = other DNA
atgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttga

SEQ ID NO: 10 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 11 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 12 - protein, 1103 aa, Homo sapiens
MTACARRAGGLPDPGLCGPAWWAPSLPRLPRALPRLPLLLLLLLLQPPALSAVFTVGVLGPWACDPIFSRARPDLAARLAAARLNRDPGLAGGPRFEVALLPEPCRTPGSLGAVSSALARVSGLVGPVNPAACRPAELLAEEAGIALVPWGCPWTQAEGTTAPAVTPAADALYALLRAFGWARVALVTAPQDLWVEAGRSLSTALRARGLPVASVTSMEPLDLSGAREALRKVRDGPRVTAVIMVMHSVLLGGEEQRYLLEAAEELGLTDGSLVFLPFDTIHYALSPGPEALAALANSSQLRRAHDAVLTLTRHCPSEGSVLDSLRRAQERRELPSDLNLQQVSPLFGTIYDAVFLLARGVAEARAAAGGRWVSGAAVARHIRDAQVPGFCGDLGGDEEPPFVLLDTDAAGDRLFATYMLDPARGSFLSAGTRMHFPRGGSAPGPDPSCWFDPNNICGGGLEPGLVFLGFLLVVGMGLAGAFLAHYVRHRLLHMQMVSGPNKIILTVDDITFLHPHGGTSRKVAQGSRSSLGARSMSDIRSGPSQHLDSPNIGVYEGDRVWLKKFPGDQHIAIRPATKTAFSKLQELRHENVALYLGLFLARGAEGPAALWEGNLAVVSEHCTRGSLQDLLAQREIKLDWMFKSSLLLDLIKGIRYLHHRGVAHGRLKSRNCIVDGRFVLKITDHGHGRLLEAQKVLPEPPRAEDQLWTAPELLRDPALERRGTLAGDVFSLAIIMQEVVCRSAPYAMLELTPEEVVQRVRSPPPLCRPLVSMDQAPVECILLMKQCWAEQPELRPSMDHTFDLFKNINKGRKTNIIDSMLRMLEQYSSNLEDLIRERTEELELEKQKTDRLLTQMLPPSVAEALKTGTPVEPEYFEQVTLYFSDIVGFTTISAMSEPIEVVDLLNDLYTLFDAIIGSHDVYKVETIGDAYMVASGLPQRNGQRHAAEIANMSLDILSAVGTFRMRHMPEVPVRIRIGLHSGPCVAGVVGLTMPRYCLFGDTVNTASRMESTGLPYRIHVNLSTVGILRALDSGYQVELRGRTELKGKGAEDTFWLVGRRGFNKPIPKPPDLQPGSSNHGISLQEIPPERRRKLEKARPGQFS

SEQ ID NO: 13 - DNA, 3312 bp, synthetic construct
  source 1..3312: mol_type = other DNA
atgacagcctgtgccaggagagctggtgggcttcctgaccctgggctctgtggtccagcttggtgggctccctccctgcccagactccccagggccctgcccaggctccctctcctgctgctcctgcttctgctgcagccccctgccctcagtgcagtgttcactgtgggggtcctgggcccctgggcttgtgaccccatcttctctagggctagacctgacctggctgccaggctggctgcagccaggctgaacagggaccctggcctggcagggggtcccaggtttgaggtagccctgctgccagagccttgcaggacaccaggctccctgggggcagtgtcctctgccctggccagagtgtcaggcctagtgggtcctgtgaaccctgcagcctgcagaccagcagagctgctggctgaagaagctgggatagcactggtgccctggggctgcccctggactcaggctgagggcaccacagcccctgcagtgaccccagctgcagatgccctctatgccctgcttagagcatttggctgggccagagtggccctggtcactgcccctcaggacctgtgggtggaggcaggaaggtcactgtccacagcactcagggccagaggcctgcctgtggcctctgtgacttccatggagcccttggacctgtctggagccagagaggccctgaggaaggttagagatgggcccagggtcacagcagtgatcatggtgatgcacagtgtgctgctgggtggagaggagcagaggtacctcctggaggctgcagaggagctgggcctgacagatggctccctggtcttcctgccctttgacaccatccactatgccttgtccccaggcccagaggccttggctgcactagccaacagctcccagcttagaagggcccatgatgcagtgctcaccctcaccagacactgtccctctgaaggctcagtgctggacagcctgagaagggctcaagagaggagagagctgccctctgacctcaatctgcagcaggtctccccactctttggcaccatctatgatgctgtcttcttgctggcaaggggagtggcagaagccagagctgctgcaggtggcagatgggtgtcaggagcagctgtggccaggcacatcagggatgcccaggtccctggcttctgtggggacctaggaggagatgaggagcccccatttgtgctgctagacacagatgctgcaggagacaggctttttgccacatacatgctggatcctgccaggggctccttcctcagtgcaggtaccaggatgcacttcccaagagggggatcagcacctggacctgaccccagctgctggtttgatccaaacaacatctgtggtggaggactggagcctggccttgtctttcttggcttcctcctggtggttgggatggggctggctggggccttcctggcccattatgtgaggcacaggctacttcacatgcaaatggtctcaggccccaacaagatcatcctgactgtggatgacatcacctttctccacccacatgggggcacctctagaaaggtggcccaggggagtagatcaagtctgggtgccaggagcatgtcagacattaggtctggccccagccaacacttggacagccccaacattggtgtctatgagggagacagggtttggctgaagaaattcccaggggatcagcacatagctatcaggccagcaaccaagacagccttctccaagctccaggagctcaggcatgagaatgtggccctctacctggggcttttcctggctaggggagcagaaggccctgcagccctctgggagggcaacctggctgtggtctcagagcactgcaccaggggctctcttcaggacctccttgctcagagagaaataaagctggactggatgttcaagtcctccctcctgctggaccttatcaagggaataaggtatctgcaccataggggagtggctcatgggagactgaagtcaagaaactgcatagtggatggcagatttgtactcaagatcactgaccatggccatgggagactgctggaagcacagaaggtgctacctgagcctcccagagctgaggaccagctgtggacagcccctgagctgcttagggacccagccctggagaggagaggaaccctggcaggagatgtctttagcttggccatcatcatgcaagaagtagtgtgcagaagtgccccttatgccatgctggagctcactcctgaggaagtggtgcagagggtgagaagtccccctccactgtgtaggcccttggtgtccatggaccaggcacctgttgagtgtatcctcctgatgaagcagtgctgggcagagcagcctgaacttagaccctccatggaccacacctttgacctgttcaagaacatcaacaagggcaggaagaccaacatcattgactcaatgcttagaatgctggagcagtactctagtaacctggaggatctgatcagggagaggacagaggagctggagctggaaaagcagaagacagacagactgcttacacagatgctgcctccttctgtggctgaggccttgaagacagggacaccagtggagcctgagtactttgagcaagtgacactgtactttagtgacattgtgggcttcaccaccatctctgccatgagtgagcccattgaggttgtggacctgctcaatgatctctacacactctttgatgccatcattggttcccatgatgtctacaaggtggagacaataggggatgcctatatggtggcctctgggctgccccagaggaatgggcagaggcatgctgcagagattgccaacatgtcactggacatcctcagtgctgtgggcactttcaggatgaggcatatgcctgaggttccagtgaggatcagaataggcctgcacagtggtccatgtgtggcaggggtggtgggcctcaccatgcccaggtactgcctgtttggggacacagtcaacactgccagtagaatggagtccactgggctgccttacagaatccatgtgaacttgagcactgtggggattctcagggctctggacagtggctaccaggtggagctgaggggcaggactgagctgaagggcaagggggcagaggacactttctggctagtgggcagaagaggcttcaacaagcccatccccaaaccccctgacctgcaaccagggtccagcaaccatggcatcagcctgcaggagatcccacctgagagaaggagaaagctggagaaggccaggccaggccagttctcttga

SEQ ID NO: 14 - DNA, 3312 bp, synthetic construct
  source 1..3312: mol_type = other DNA
atgacagcctgtgccagaagggcaggtgggcttccagacccagggctctgtggtcctgcttggtgggctccctccctgcccagactccccagagccctgcccaggctccccctcctgctgctcctgcttctgctgcagcccccagccctcagtgctgtgttcacagtgggggtcctgggcccctgggcttgtgaccccatcttctctagggctaggcctgacctggcagccaggctggcagctgccagactgaacagggaccctggcctggcaggaggtcccaggtttgaggtagctctgctgccagagccttgcagaacacctggcagtctgggggctgtgtccagtgcactggccagagtgtcaggcttggtgggtcctgtgaaccctgcagcctgcagaccagctgagctgctggctgaagaagctgggattgctctggtgccctggggctgcccctggacccaggctgagggcaccacagcccctgctgtgaccccagctgcagatgccctctatgccctgcttagggcatttggctgggccagggtggccctggtcacagcaccccaggacctgtgggtggaggctggaaggtcactgtccactgcactcagggccaggggcctgcctgtggcctcagtgacttccatggagcccttggacctgtctggagccagggaggccctgaggaaggttagagatgggcccagggtcacagcagtgatcatggtgatgcacagtgtgctgctgggtggtgaggagcagaggtacctcctggaggctgcagaggagctgggcctgacagatggctccctggtcttcctgccctttgacaccatccactatgccttgtccccaggccctgaggccttggctgcactggccaacagctcccagcttagaagggcccatgatgctgtgctcaccctcactagacactgtccctctgaaggcagtgtgctggacagcctgagaagggctcaagagagaagggagctgccctctgacctcaatctgcagcaggtctccccactctttggcaccatctatgatgctgtcttcttgctggcaaggggtgtggcagaagccagagctgctgcaggtggcagatgggtgtctggagcagctgtggccaggcacatcagggatgcacaggtccctggcttctgtggggacttgggaggagatgaggagcccccatttgtgctgctggacacagatgctgcaggagacagactttttgccacatacatgctggatcctgccaggggctccttcctctctgctggtaccagaatgcacttccctagagggggatcagcacctggacctgacccctcatgctggtttgatccaaacaacatctgtggtggaggactggagccaggccttgtctttcttggcttcctcctggtggttgggatggggctggctggggccttcctggcccattatgtgaggcacaggttgcttcacatgcaaatggtctcaggccccaacaagatcatcctgactgtggatgacatcacctttctccacccacatgggggcacctctagaaaggtggcccaggggagtagatcaagtctgggtgccagaagcatgtcagacattaggagtggccccagccaacacttggacagccccaacattggtgtctatgagggagacagggtttggctgaagaaattcccaggggatcagcacatagctatcagaccagcaaccaagactgccttctccaagctccaggagctcagacatgagaatgtggccctctacctggggcttttcctggctaggggagcagaaggccctgctgccctctgggagggcaacctggctgtggtctcagagcactgcactagaggctctcttcaggacctccttgctcagagagaaataaagctggactggatgttcaagtcctccctcctgctggaccttatcaagggaataaggtatctgcaccataggggtgtggctcatgggaggctgaagtcaagaaactgcatagtggatggcagatttgtactcaagatcactgaccatggccatgggagactgctggaagcacagaaggtgctgccagagcctcccagagcagaggaccagctgtggacagcccctgagctgcttagggacccagccctggagagaaggggaacactggctggagatgtctttagcttggccatcatcatgcaagaagtagtgtgcaggagtgccccttatgccatgctggagctcactccagaggaagtggtgcagagggtgagaagcccacctccactgtgtagacccttggtgtccatggaccaggcacctgtggagtgtatcctcctgatgaagcagtgctgggcagagcagcctgaacttaggccctccatggaccacacctttgacctgttcaagaacatcaacaagggcagaaagaccaacatcattgactcaatgcttagaatgctggagcagtactctagtaacctggaggatctgatcagggagaggactgaggagctggagctggaaaagcagaagacagacagactgcttacacagatgctgcctccctctgtggctgaggccttgaagacagggacaccagtggagcctgagtactttgagcaagtgacactgtactttagtgacattgtgggcttcaccaccatctctgccatgagtgagcccattgaggttgtggacctgctcaatgatctctacacactctttgatgccatcattggttcccatgatgtctacaaggtggagacaataggggatgcctatatggtggcctctgggctgccccagaggaatgggcagaggcatgctgcagagattgccaacatgtcactggacatcctcagtgctgtgggcactttcaggatgagacatatgcctgaggttcctgtgaggatcaggataggcctgcactctggtccatgtgtggcaggagtggtgggcctcaccatgcctagatactgcctgtttggggacacagtcaacacagcctccaggatggagtccacagggctgccttacaggatccatgtgaacttgagcactgtggggattctcagggctctggactcaggctaccaggtggagctgaggggcaggactgagctgaagggcaagggagctgaggacactttctggcttgtgggcagaaggggcttcaacaagcccatccccaaaccacctgacctgcaaccagggtccagcaaccatggcatcagcctgcaggagatcccacctgagaggagaaggaagctggagaaggcaaggccaggccagttctcttga